(19) United States
(12) Patent Application Publication
(10) Pub. No.: US 2018/0207202 A1
(43) Pub. Date: Jul. 26, 2018

(54) METHODS FOR MOBILIZING HEMATOPOIETIC STEM CELLS

(71) Applicant: Ohio State Innovation Foundation, Columbus, OH (US)

(72) Inventors: Jianhua Yu, Columbus, OH (US); Steven Devine, Columbus, OH (US); Michael A. Caligiuri, Columbus, OH (US); Shun He, Malden, MA (US)

(73) Assignee: Ohio State Innovation Foundation, Columbus, OH (US)

(21) Appl. No.: 15/860,612

(22) Filed: Jan. 2, 2018

Related U.S. Application Data
(62) Division of application No. 14/761,181, filed on Jul. 15, 2015, now abandoned.
(60) Provisional application No. 61/752,735, filed on Jan. 15, 2013.

(51) Int. Cl. A61K 35/28; A61K 45/06; A61K 31/395; A61K 38/18

(52) U.S. Cl. CPC A61K 35/28; A61K 45/06; A61K 31/395; A61K 2035/124; A61K 38/18

(57) ABSTRACT

Disclosed are methods of mobilizing hematopoietic cells in a subject by administering to the subject a combination of Flt3 ligand (FLT3L) and a cell adhesion inhibitor. Also disclosed are methods of mobilizing hematopoietic cells in a cell culture population by contacting the cell culture population with Flt3 ligand. Mobilized hematopoietic cells can subsequently be harvested and used for hematopoietic cell transplantation. Further disclosed are compositions comprising a FLT3L, compositions comprising FLT3L and a cell adhesion inhibitor, and composition comprising FLT3L and Plerixafor.

### CROSS-REFERENCE TO RELATED APPLICATIONS

This application is a divisional under 35 U.S.C. § 120 of U.S. application Ser. No. 14/761,181, filed Jul. 15, 2015, which is a national phase entry under 35 U.S.C. § 371 of International Application No. PCT/US2014/011628, filed Jan. 15, 2014, which claims the benefit under 35 U.S.C. § 119(e) of U.S. Provisional Application No. 61/752,735, filed Jan. 15, 2013, the contents of each of which are hereby incorporated by reference herein in their entireties.

### STATEMENT REGARDING FEDERALLY SPONSORED RESEARCH OR DEVELOPMENT

This invention was made with government support under ROI CA155521 awarded by the National Institutes of Health. The government has certain rights in the invention.

### BACKGROUND

Hematopoietic stem cell transplantation (HSCT) has curative potential for patients with hematologic malignancies through destroying tumor cells by irradiation and graft-versus-tumor (GVT) effects. However, HSCT is associated with complications such as graft-versus-host disease (GVHD), infection, and relapse, which result in significant morbidity and mortality. The sources of grafts can be peripheral blood stem cells (PBSC), bone marrow (BM), and cord blood. Granulocyte colony stimulating factor (G-CSF) is routinely used to mobilize PBSC. Due to ease of procurement and donor convenience, G-CSF-mobilized-PBSC (G-PBSC) have been the most commonly used donor graft source, and peripheral blood stem cell transplant (PBSCT) has become the predominant graft source over the last two decades, accounting for approximately 75% of allogeneic and 100% of autologous transplants. This makes mobilization and collection of blood stem cells a critical part of the whole transplantation procedure. However, the trend to use PBSC for allogeneic transplantation may have been premature as recent data suggest G-PBSC transplantation is associated with higher rates of chronic GVHD and worse survival in children and adults with aplastic anemia. In addition, some normal donors (from 5-10%) mobilize stem cells poorly in response to G-CSF, and many suffer significant bone pain related to it. Further, from 10-30% of autologous transplant patients (varied depending on disease types and age) respond poorly to G-CSF or G-CSF plus chemotherapy. For this reason, Plerixafor has been used in combination with G-CSF to mobilize peripheral blood stem cells. However, the combination of Plerixafor and G-CSF is still not effective for all patients, including those who have failed a previous mobilization, and it is reported that the success rate of the combination for people who experienced a previous failure for mobilization is about 70%. In addition, compared to G-CSF alone, mobilization with the Plerixafor and G-CSF combination results in a smaller proportion of CD34⁺ stem cells, although total CD34⁺ cells are significantly increased. Moreover, the combination of Plerixafor and G-CSF increases circulating tumor cells in acute myelogenous leukemia and plasma cell leukemia patients, so it is not recommended for HSC mobilization in leukemia patents.

Because hematological malignancies are among the most commonly diagnosed forms of cancer, what is needed in the art are better methods and compositions for mobilizing hematopoietic cells that can be harvested and used to improve the efficiency, efficacy, and safety of hematopoietic cell transplantation.

In accordance with the purpose(s) of the invention, as embodied and broadly described herein, the invention, in one aspect, relates to methods of mobilizing hematopoietic cells in a subject or a cell culture population.

Disclosed herein is a method of mobilizing hematopoietic cells in a subject, consisting essentially of administering to a subject an effective amount of FLT3L and a cell adhesion inhibitor, whereby administering the effective amount of FLT3L and the cell adhesion inhibitor mobilizes hematopoietic cells in the subject.

Disclosed herein is a method of mobilizing hematopoietic cells in a cell culture population, consisting essentially of contacting a cell culture population with an effective amount of FLT3L, whereby contacting the cell culture population with the effective amount of FLT3L mobilizes hematopoietic cells in the cell culture population.

Disclosed herein is a method of mobilizing hematopoietic cells in a subject comprising administering to a subject an effective amount of FLT3L and a cell adhesion inhibitor, whereby administering the effective amount of FLT3L and the cell adhesion inhibitor mobilizes hematopoietic cells in the subject.

Disclosed herein is a method of mobilizing hematopoietic cells in a cell culture population comprising contacting a cell culture population with an effective amount of FLT3L, whereby contacting the cell culture population with the effective amount of FLT3L mobilizes hematopoietic cells in the cell culture population.

Disclosed herein is a method of treating a subject in need of hematopoietic cells comprising administering to a subject an effective amount of hematopoietic cells mobilized in and harvested from a donor, wherein the donor was treated according to a method comprising administering to the donor an effective amount of FLT3L and a cell adhesion inhibitor, whereby administering the effective amount of FLT3L and the cell adhesion inhibitor mobilizes hematopoietic cells in the donor.

Disclosed herein is a method of mobilizing hematopoietic cells in a subject comprising administering to a subject an effective amount of a composition comprising FLT3L, whereby administering the effective amount of the composition mobilizes hematopoietic cells in the subject.

Disclosed herein is a method of mobilizing hematopoietic cells in a subject comprising administering to a subject an effective amount of a composition comprising FLT3L and Plerixafor, whereby administering the effective amount of the composition mobilizes hematopoietic cells in the subject.

Disclosed herein is a method of mobilizing hematopoietic cells in a subject comprising (a) administering to a subject an effective amount of a composition comprising FLT3L, and (b) administering to the subject an effective amount of a composition comprising Plerixafor, whereby administering an effective amount of the composition of step (a) and the composition of step (b) mobilize hematopoietic cells in the subject.

Disclosed herein is a method of mobilizing hematopoietic cells in a subject comprising administering to a subject an effective amount of a composition comprising FLT3L, administering to the subject an effective amount of a composition comprising Plerixafor, and mobilizing hematopoietic cells in the subject. In an aspect, the mobilized hematopoietic cells can be hematopoietic stem cells.

Disclosed herein is a method of treating a subject in need of hematopoietic cells comprising administering to a subject an effective amount of hematopoietic cells mobilized in and harvested from a donor, wherein the donor was treated according to a method comprising administering to the donor an effective amount of a composition comprising FLT3L and a cell adhesion inhibitor, whereby administering the composition mobilizes hematopoietic cells in the donor.

Disclosed herein is a method of treating a subject in need of hematopoietic cells comprising administering to a subject an effective amount of hematopoietic cells mobilized in and harvested from a donor, wherein the donor was treated according to a method comprising administering to the donor an effective amount of a composition comprising FLT3L and a composition comprising Plerixafor, whereby administering the composition mobilizes hematopoietic cells in the donor.

Disclosed herein is a composition comprising FLT3L and a cell adhesion inhibitor.

Disclosed herein is a composition comprising FLT3L and Plerixafor.

### BRIEF DESCRIPTION OF THE FIGURES

The accompanying Figures, which are incorporated in and constitute a part of this specification, illustrate several aspects and together with the description serve to explain the principles of the invention.

FIGS. 1(A and B) shows that a combination of Flt3 ligand (FLT3L) and Plerixafor mobilized hematopoietic stem and precursor cells.

FIGS. 2(A and B) shows that FLT3L and the combination of FLT3L plus Plerixafor expanded NK-cells.

FIG. 3 shows that the proportion of CD8⁺ T cells decreased in peripheral blood stem cell mobilization by FLT3L and the combination of FLT3L plus Plerixafor.

FIG. 4 shows that co-administration of a cytokine, in particular INF-β, suppressed stem cell mobilization by FLT3L and is therefore contraindicated for use with the disclosed methods.

FIG. 5 shows survival data of irradiation protection in (A) a syngeneic model (C57BL/6), and (B) against GVHD in a fully mismatched model (C57BL/6 to BALB/c).

FIG. 6(A-D) shows that the FP combination effectively mobilized LSK cells into peripheral blood.

FIG. 7(A-D) shows that the FP combination effectively mobilized NK cells, Tregs, and DCs into peripheral blood.

FIG. 8 shows that FP-mobilized grafts significantly enhanced survival of mice following both syngeneic transplantation (A) and allogeneic transplantation (B).

FIG. 9 shows the flow cytometric analysis of Lin⁻ (lineage negative) cells in peripheral blood mobilized by different regimens.

FIG. 10 shows the white blood cell counts in peripheral blood mobilized by different regimens.

FIG. 11 shows the synergistic effect of the FP combination on LSK cell mobilization.

FIG. 12 shows that neutrophils comprised the majority of cells in the peripheral blood mobilized by the GP combination.

FIG. 13 shows the synergistic effect of the FP combination on the colony forming units in mobilized cells.

FIG. 14(A-C) shows the frequency of Treg cells in the peripheral blood mobilized by different regimens.

FIG. 15 shows the LSK flow cytometric analysis of the mice with syngeneic transplantation of mobilized peripheral blood cells depleted of red blood cells.

FIG. 16 shows that mice receiving allogeneic grafts mobilized by the FP combination had both long-term HSCs and short-term HSCs in bone marrow.

### DETAILED DESCRIPTION

The present invention can be understood more readily by reference to the following detailed description of the invention, the figures and the examples included herein.

Before the present compositions and methods arc disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

Definitions

As used in the specification and the appended claims, the singular forms “a,” “an” and “the” include plural referents unless the context clearly dictates otherwise. Thus, for example, reference to “a cell adhesion inhibitor,” “an amount,” or “the subject” includes mixtures of two or more such cell adhesion inhibitors, amounts, or subjects, and the like.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “subject” refers to the target of administration, e.g., an animal. Thus the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In an aspect, a subject is a mammal. In another aspect, a subject is a human. The term does not denote a particular age or sex. Thus, adult, child, adolescent and newborn subjects, as well as fetuses, whether male or female, are intended to be covered.

In an aspect of the disclosed methods, a subject can be diagnosed with a need for mobilizing hematopoietic cells prior to the administering step. For example, the subject can be diagnosed with a need for hematopoietic stem cells. In an aspect of the disclosed methods, the subject can be diagnosed with a need for autologous hematopoietic stem cell transplantation. In an aspect of the disclosed methods, the subject is a donor for an allogeneic hematopoietic stem cell transplant.

As used herein, the term “patient” refers to a subject afflicted with a disease or disorder. A patient can include human and veterinary subjects. In an aspect of the disclosed methods, the patient can be diagnosed with a need for treatment of one or more disorders or diseases prior to the administering step.

As used herein, the term “recipient” refers to a subject or patient who receives treatment according to the disclosed methods or a hematopoietic cell or hematopoietic stem cell transplant, regardless of whether the transplant is allogeneic or autologous. The terms “subject,” “patient,” and “recipient” can be used interchangeably throughout the specification.

As used herein, the term “donor” refers to a source of mobilized hematopoietic cells. Thus, a donor according to the disclosed methods can be a mammal, for example, a human.

As used herein, the term “treatment” refers to the medical management of a patient with the intent to cure, ameliorate, stabilize, or prevent a disease, pathological condition, or disorder. This term includes the use for aesthetic and self-improvement purposes; for example, such uses include, but are not limited to, the use of the claimed methods for improving circulation, red blood cell count, energy level, or sense of well-being. This term includes active treatment directed specifically toward the improvement of a disease, pathological condition, or disorder; causal treatment directed toward removal of the cause of the associated disease, pathological condition, or disorder; palliative treatment designed for the relief of symptoms rather than the curing of the disease, pathological condition, or disorder; preventative treatment directed to minimizing or partially or completely inhibiting the development of the associated disease, pathological condition, associated symptoms or disorder; and supportive treatment employed to supplement another specific therapy directed toward the improvement of the associated disease, pathological condition, associated symptoms or disorder. Thus, the term covers any treatment of a subject and includes: (i) preventing a disease from occurring in a subject that can be predisposed to the disease but has not yet been diagnosed as having it; (ii) inhibiting the disease, i.e., arresting its development; or (iii) relieving the disease, i.e., causing regression of the disease.

As used herein, the phrase “identified to be in need of treatment for a disorder,” “in need of treatment,” or the like, refers to selection of a subject based upon need for treatment of the disorder. For example, a subject can be identified as having a need for treatment of a disorder (e.g., a disorder related to hematological malignancies) based upon an earlier diagnosis by a person of skill and thereafter subjected to treatment for the disorder. It is contemplated that the administration can, in an aspect, be performed by a person different from the person making the diagnosis. It is also contemplated that a diagnosis can be made by one who subsequently performs the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, intra-urethral administration, ophthalmic administration, intra-aural administration, intra-cerebral administration, intra-thecal administration, rectal administration, sublingual administration, buccal administration, intra-peritoneal administration and parenteral administration, including intravenous administration, intra-arterial administration, intramuscular administration, intradermal administration and subcutaneous administration. Administration can be continuous, by bolus or intermittent. In various aspects, a preparation can be administered therapeutically to treat an existing disease or condition. In an aspect, a preparation can be administered prophylactically for prevention of a disease or disorder.

The term “contacting” as used herein refers to bringing a disclosed compound or disclosed composition, for example FLT3L or a composition comprising FLT3L or a composition comprising FLT3L and a cell adhesion inhibitor such as Plerixafor, and a cell culture population or other biological entity together in such a manner that the compound can affect the activity of the cell culture population, either directly, i.e., by interacting with a cell culture population, or indirectly, i.e., by interacting with another molecule, co-factor, factor, or protein on which the activity of the cell culture population is dependent. For example, a cell culture population can be contacted with FLT3L for the purpose of mobilizing hematopoietic cells.

As used herein, the term “contemporaneously” refers to administration to a subject or contacting a cell culture population with two or more disclosed compounds or disclosed compositions at or around the same time. For example, contemporaneously can mean that FLT3L and a cell adhesion inhibitor can be administered at the same time; FLT3L can be administered immediately before a cell adhesion inhibitor; or FLT3L can be administered substantially before a cell adhesion inhibitor. For example, FLT3L can be administered from about 1 hour to about 24 hours before a cell adhesion inhibitor is administered. In an aspect, a cell adhesion inhibitor can be Plerixafor.

As used herein, the terms “effective amount” and “amount effective” refer to an amount of a composition that is sufficient to achieve a desired result or to have an effect on an undesired condition. For example, a “therapeutically effective amount” refers to an amount of a composition that is sufficient to achieve a desired therapeutic result or to have an effect on undesired symptoms in a patient but is generally not so large as to cause adverse side effects. The specific therapeutically effective dose level of a composition for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed; and like factors well known in the medical arts. For example, a person of ordinary skill in the art, for example a physician, would know that the dose (i.e., the amount) of a compound can be started at levels lower than those required to achieve a desired therapeutic effect and to gradually increase the dose until a desired effect is achieved. If desired, the effective daily dose call be divided into multiple doses for purposes of administration. Consequently, single dose compositions can contain such amounts or submultiples thereof to make up the daily dose. The dosage can be adjusted by the individual physician in the event of any contraindications. Dosage can vary and can be administered in one or more dose administrations daily, for one or several days. Guidance can be found in the literature for appropriate dosages for various classes of pharmaceutical products. Furthermore, a preparation can be administered in a “prophylactically effective amount” that is effective for prevention of a disease or disorder. As used herein, the terms “compound,” “agent,” “composition” and “preparation” can be used interchangeably.

The term “pharmaceutically acceptable” describes a material that is not biologically or otherwise undesirable, i.e., without causing an unacceptable level of undesirable biological effects or interacting in a deleterious manner.

The term “hematopoietic cells” describes all bone marrow-derived cell types and non-bone marrow-derived cell types in the blood. This term refers to mature cell types and their immature precursors, or hematopoietic stem cells, that are identifiable either by morphology or, mostly, by a distinct pattern of cell surface markers. The term is used to distinguish these cells from other cell types found in the body and also includes T-cells and distinctive subsets, which are the only hematopoietic cells that are not generated in the bone marrow. Hematopoietic cells are subgrouped broadly into myeloid cells, which include leukocytes and erythrocytes, and lymphoid cells. Examples of hematopoietic cells include, but are not limited to, hematopoietic stem cells, basophilic myelocytes, basophils, B-cells, BFU-E, BFU-Mk, CFU-Bas, CFU-E, CFU-Eo, CFU-G, CFU-GEMM, CFU-GM, CFU-Mk, common dendritic progenitor cells, common lymphoid progenitor cells, common myeloerythroid progenitor cells, common myeloid progenitor cells, common myelolymphoid progenitor cells, DN1 cells, DN2 cells, DN3 cells, DN4 cells, double-positive cells (DP cells), eosinophilic myelocytes, eosinophils, erythrocytes, lymphoid stem cells, lymphoid-related dendritic cells, macrophages, mast cells, megakaryocytes, memory B-cells, memory cells, memory T-cells, monoblasts, monocytes, myeloblasts, myeloid stem cells, myeloid-related dendritic cells, neutrophilic myelocytes, neutrophils, NK-cells, NKT-cells, platelets, pro-B1-cells, pro-B-2-cells, pro-B-cells, proerythroblasts, erythroblasts, reticulocytes, promonocytes, regulatory T-cells (Tregs), T-cells, T-helper cells, Th0 cells, Th1 cells, Th2 cells, Th3 cells, Th17 cells, dendritic cells, myeloid-derived suppressor cells (MDSC), M1 macrophages, and M2 macrophages.

The term “cell adhesion inhibitor” describes small molecules and macromolecules that prevent cell adhesion. Cell adhesion is a process by which cells associate with each other, migrate towards a specific target or localize within the extracellular matrix of various tissues in a subject. For example, cell adhesion is responsible for the adhesion of hematopoietic cells to endothelial cells, which line blood vessels, and the subsequent migration of those hematopoietic cells out of blood vessels and to a site of injury. Thus, cell adhesion inhibitors prevent this kind of contact between cells. Examples of cell adhesion inhibitors include, but are not limited to, Plerixafor, CTCE-9908, thiopseudourea compounds, TN14003, KRH-2731, KRH-3955, TC14012, AMD070, BKT-140, neutrophil elastase, cathepsin G, and derivatives thereof.

The term “derivative” refers to a compound having a structure attained from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound. For example, it is known in the art that a derivative may be synthesized by adding a methyl, acetate, or other side chain to a parent structure in such a way that the functionality of the molecule is effectively unchanged.

The term “cell culture population” refers to at least one cell or multiple cells grown in in vitro or ex vivo culturing conditions. The term is meant to encompass any type of cell that can be cultured in vitro or ex vivo. Examples include, but are not limited to, bone marrow cells, embryonic stem cells, umbilical cord blood cells, induced pluripotent stem cells (iPS), hematopoietic stem cells, hematopoietic progenitor cells and peripheral blood cells. In an aspect, a cell culture population can comprise additional growth factors, cytokines, feeder cells, and cell support structures. In an aspect, a cell culture population can be maintained in a 2D culture, 3D culture or a bioreactor.

The terms “expand” or “expands” refer to growing cells under culturing conditions. This can include in vitro and ex vivo cultures and can comprise any means known in the art for providing the necessary conditions such that a given cell type can survive, grow, or multiply.

As used herein, the term “determining” can refer to measuring or ascertaining a quantity or an amount or a change in expression and/or in activity level or in prevalence. For example, determining can refer to measuring or ascertaining the quantity or amount of mobilized cells, such as tregs or NKs or CD8⁺ cells or neutrophils or white blood cells or hemopoietic cells or stem cells. Methods and techniques used to determining the amount or quantity or prevalence of a disclosed transcript or polypeptide or cell in a sample as used herein can refer to the steps that the skilled person would take to measure or ascertain some quantifiable value of the transcript or polypeptide or cell in the sample. The art is familiar with the ways to measure an amount of a disclosed cell type.

Hematopoietic Cell Mobilization

Flt3 ligand (FLT3L) is a stem cell-specific growth factor that expands and mobilizes stern cells after administration for about ten days. FLT3L can be administered either as a single agent or in combination with other molecules such as IL-8 and G-CSF.

In order to reduce transplantation complications and to promote prompt hematopoietic reconstitution in patients, what is needed in the art is a method for rapidly obtaining a stem cell graft, with high numbers of stern and progenitor cells along with favorable immunomodulatory subsets. Of particular interest would be a method capable of mobilizing so-called natural killer or NK cells, which are capable of destroying tumor cells, clearing infection and reducing or preventing graft versus host disease (GVHD).

Therefore, disclosed herein is the surprising discovery that a method using a combination of FLT3L and Plerixafor can mobilize hematopoietic cells for transplantation better than a method using G-CSF alone or G-CSF in combination with Plerixafor.

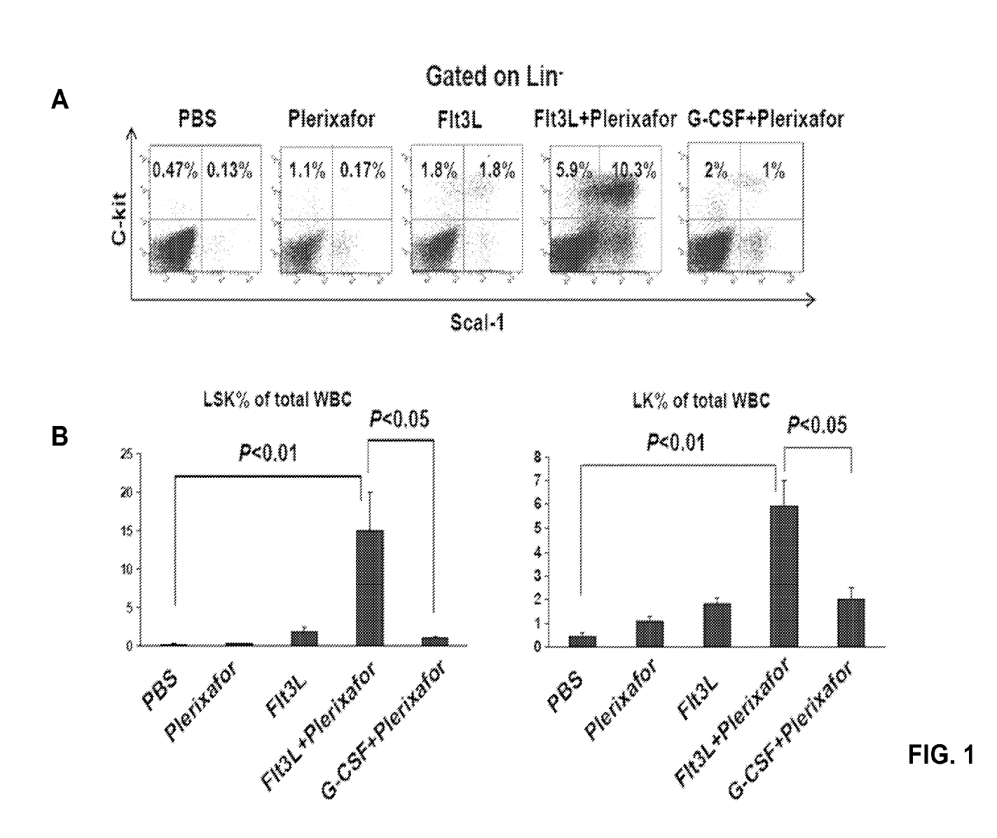

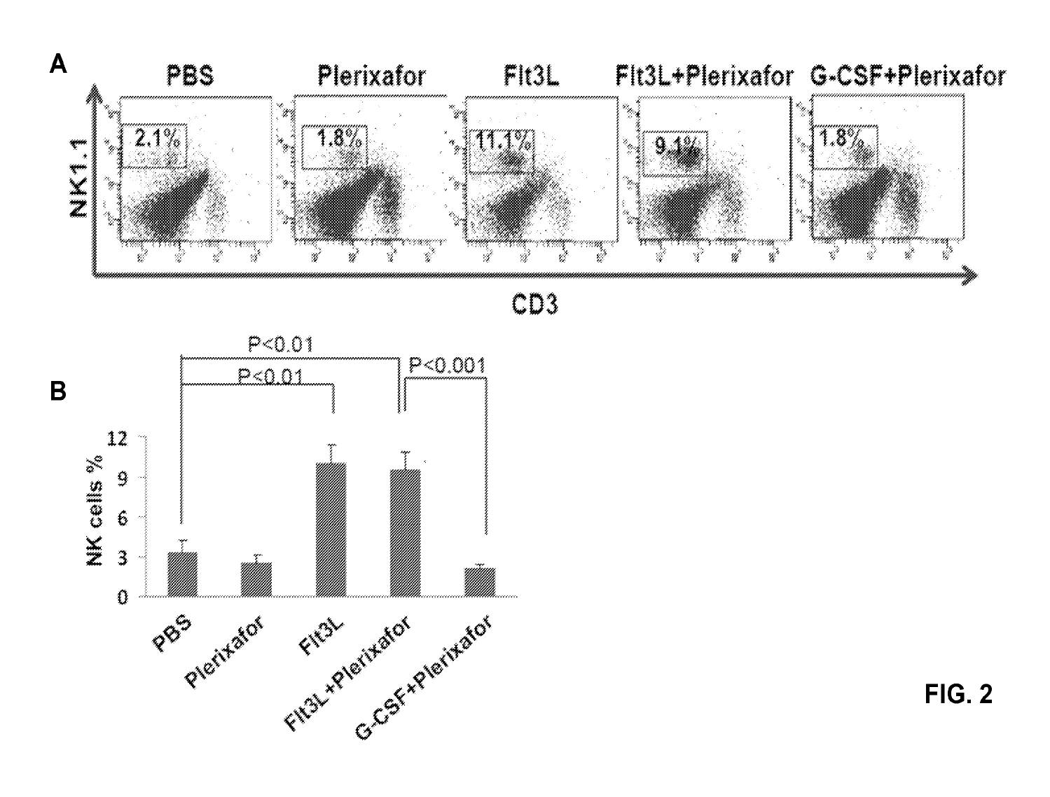

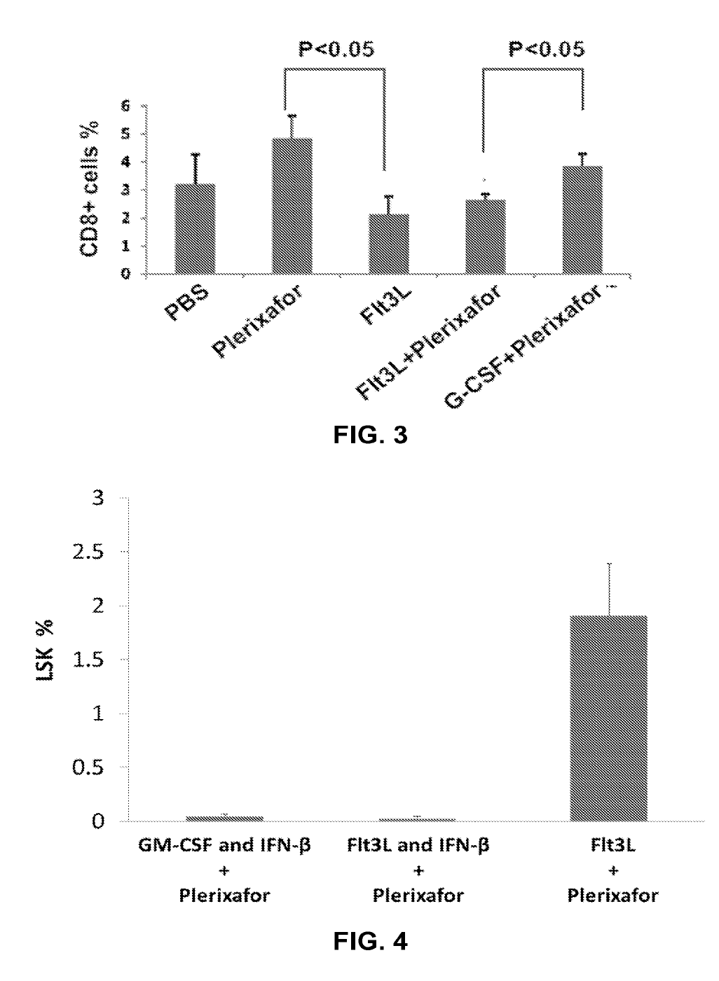

The data presented herein demonstrate that the combination of FLT3L and Plerixafor increased the number of stem and precursor cells (FIG. 1), proportionally raises the number of NK cells (FIG. 2), and decreases CD8⁺ T cells (FIG. 3). NK cells have been shown to decrease relapse and reduce GVHD, while donor CD8⁺ T cells are a major promoter of GVHD. These findings demonstrate a more effective mobilization means for peripheral blood stem cell transplantation compared to current clinical standards. The disclosed methods result in a rapid reconstitution of desirable cells and a reduction of complications such as GVHD, disease relapse and infection. FLT3L expands hematopoietic stem/precursor cells and NK cells, which are subsequently mobilized by Plerixafor. Therefore, the combination of FLT3L and Plerixafor is synergistic and mobilizes more stem/precursor cells and NK cells compared to currently used clinical methods.

Disclosed is a method of mobilizing hematopoietic cells in a subject consisting essentially of administering to a subject an effective amount of FLT3L and a cell adhesion inhibitor, whereby administering the effective amount of FLT3L and the cell adhesion inhibitor mobilizes hematopoietic cells in the subject. In an aspect, the hematopoietic cells can be hematopoietic stem cells. In an aspect, the disclosed method can exclude the administration of INF-β.

In an aspect, FLT3L can be administered to a subject once a day for from about 5 days to about 15 days. For example, FLT3L can be administered to a subject once a day for 5, 6, 7, 8, 9, 10, 11, 12, 13, 14 or 15 days, or any time in between. Moreover, FLT3L can be administered to a subject twice a day for from about 5 days to about 15 days. For example, FLT3L can be administered twice a day for 5, 6, 7, 8, 9, 10, 11, 12, 13, 14 or 15 days, or any time in between.

In an aspect, FLT3L can be administered to a subject in an amount from about 0.01 mg/kg to about 1.0 mg/kg, or any amount in between. Thus, the amount of FLT3L administered to a subject in mg/kg can be 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1.0. In an aspect, the amount of FLT3L administered to a subject can be about 0.35 mg/kg.

In an aspect, FLT3L and a cell adhesion inhibitor can be administered contemporaneously. For example, FLT3L can be administered at the same time with or from about 1 hour to about 24 hours before a cell adhesion inhibitor is administered to a subject. Thus, FLT3L can be administered 1, 2, 3, 4, 5, 6, 7, 8, 9, 10, 11, 12, 13, 14, 15, 16, 17, 18, 19, 20, 21, 22, 23 or 24 hours, or any time in between, before a cell adhesion inhibitor is administered to a subject.

In an aspect, FLT3L can be administered from about 1 day to about 15 days before a cell adhesion inhibitor is administered to a subject. For example, FLT3L can be administered 1, 2, 3, 4, 5, 6, 7, 8, 9, 10, 11, 12, 13, 14 or 15 days, or any time in between, before a cell adhesion inhibitor is administered to a subject.

In an aspect, a cell adhesion inhibitor can be one or more of Plerixafor, CTCE-9908, a thiopseudourea compound, TN14003, KRH-2731, KRH-3955, TC14012, AMD070, BKT-140, neutrophil elastase, cathepsin G, and derivatives thereof. It is contemplated that other cell adhesion inhibitors, for example any inhibitor of CXCR4, can be used in the disclosed methods.

In an aspect, a cell adhesion inhibitor can be Plerixafor. Plerixafor is a macrocyclic compound antagonist of the alpha chemokine receptor CXCR4 and can be used for hematopoietic stem cell (HSC) mobilization. The SDF-CXCL12/CXCR4 retention axis disruption by this agent in the bone marrow can release a whole host of progenitor cells without the necessity of priming.

In an aspect, Plerixafor can be administered to a subject in an amount from about 0.01 mg/kg to about 1.0 mg/kg, or any amount in between. Thus, the amount of Plerixafor administered to a subject in mg/kg can be 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1.0. In an aspect, the amount of Plerixafor administered to a subject can be about 0.24 mg/kg.

Mobilized hematopoietic cells, for example hematopoietic stem cells, can be harvested from a subject via peripheral venous access or a central line. Cells can also be collected by an apheresis machine, continuous-flow blood cell separator, or a comparable device. Other methods well known in the art can also be used for harvesting cells. Harvested cells can be clinical grade materials that are immediately ready for hematopoietic cell or hematopoietic stem cell transplantation. Following mobilization and harvesting of hematopoietic cells according to the disclosed methods, harvested cells can be preserved and expanded by methods well known in the art.

In an aspect, mobilized hematopoietic cells can be harvested from a subject from about 1 hour to about 36 hours after a cell adhesion inhibitor, for example Plerixafor, is administered to a subject. For example, in an aspect, hematopoietic stem cells can be harvested from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 hours, or any time in between, after a cell adhesion inhibitor is administered to a subject.

Disclosed herein is a method of mobilizing hematopoietic cells in a cell culture population consisting essentially of contacting a cell culture population with an effective amount of FLT3L, whereby contacting the cell culture population with the effective amount of FLT3L mobilizes hematopoietic cells in the cell culture population. In an aspect, the mobilized hematopoietic cells can be hematopoietic stem cells. In an aspect, the disclosed method can exclude contact with INF-β.

In an aspect, a cell culture population comprises one or more of bone marrow cells, embryonic stem cells, umbilical cord blood cells, induced pluripotent stem cells, hematopoietic stem cells, hematopoietic progenitor cells and peripheral blood cells. A cell culture population can optionally also comprise hematopoietic cells that were harvested from a subject whose hematopoietic cells had been mobilized according to the disclosed methods.

In an aspect, a cell culture population can be contacted with FLT3L once a day for from about 5 days to about 15 days. For example, a cell culture population can be contacted with FLT3L once a day for 5, 6, 7, 8, 9, 10, 11, 12, 13, 14 or 15 days, or any time in between. Moreover, a cell culture population can be contacted with FLT3L twice a day for from about 5 days to about 15 days. For example, a cell culture population can be contacted with FLT3L twice a day for 5, 6, 7, 8, 9, 10, 11, 12, 13, 14 or 15 days, or any time in between.

In an aspect, the amount of FLT3L contacting a cell culture population can be from about 0.01 μg/mL to about 1.0 μg/mL or any amount in between. Thus, the amount of FLT3L contacting a cell culture population in μg/mL can be 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1.0.

Mobilized hematopoietic cells can be harvested from a cell culture population by multiple methods known in the art including, but not limited to, flow cytometry and cell sorting, including cell sorting using magnetic beads, or by commercially available isolation kits. In an aspect, hematopoietic cells can be harvested from about 0.5 hour to about 36 hours, or any time in between, after a cell culture population is contacted with FLT3L. In an aspect, hematopoietic stem cells can be harvested from about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 hours, or any time in between, after a cell adhesion inhibitor contacts the cell culture population.

The disclosed methods can be used to expand and mobilize hematopoietic cells in a closed system at an outpatient facility. Harvested hematopoietic cells can be cleansed and concentrated by density gradient centrifugation or other means known in the art and can be available for immediate use or can be cryogenically stored for later use. Cryogenic preservation methods are well known in the art. In an aspect, harvested hematopoietic cells, after being prepared using aseptic technique, can be stored in refrigerators or freezers commonly used at points of care facilities for a period of time up to about 7 days. Furthermore, as is known in the art, harvested cells can be stored at −80° C. for several years or in liquid nitrogen indefinitely.

Harvested hematopoietic cells can further be enriched differentially based on surface antigens expressed by certain types of hematopoietic cells, e.g., using FACS, so that the fractions of the different kinds of hematopoietic cells can be isolated. In an aspect, cells can be sorted by mixing with magnetic beads coated with monoclonal antibodies against a cell surface antigen characteristically expressed by specific hematopoietic cells. Other methods well known in the art can be used to alter the number of cell types or isolate specific cell types from within a population of harvested hematopoietic cells.

In another aspect, harvested hematopoietic cells can be placed in pharmaceutically acceptable carriers with formulations well known in the art for intravenous administration. The cells can be formulated in a solution with a pH from about 6.5 to about 8.5. For examples, harvested cells can be formulated in a solution with a pH of 6.5, 6.75, 7.0, 7.25, 7.5, 7.75, 8.0, 8.25 or 8.5, or any pH value in between. Excipients can be added to make a solution isotonic. Examples of such excipients include, but are not limited to, 4.5% mannitol, normal 0.9% saline, sodium phosphate, dextrose, boric acid, sodium tartrate, propylene glycol and other inorganic or organic solutes, antioxidants, buffers, bacteriostats, suspending agents, solubilizers, thickening agents, stabilizers, preservatives, and adjuvants.

Disclosed herein is a method of treating a subject in need of hematopoietic cells mobilized according to the methods herein. Mobilized hematopoietic cells or hematopoietic stem cells are needed in the treatment of many blood disorders as well as other diseases. Blood disorders include, but are not limited to, hematological malignancies, anemias, immunodeficiencies, and ischemia, for example, limb ischemia. Other diseases and disorders that can be treated using hematopoietic cell transplants prepared according to the disclosed methods include, but are not limited to, acute lymphoblastic leukemia, acute myelogenous leukemia, chronic lymphocytic leukemia, chronic myelogenous leukemia, chronic neutrophilic leukemia, chronic eosinophilic leukemia, acute monocyctic leukemia, Hodgkin's lymphoma, non-Hodgkin's lymphoma, Waldenstrom's macroglobulinemia, multiple myeloma, primary myelofibrosis, polycythemia vera, capillary leak syndrome, hematopoietic ulcer, sickle cell anemia, aplastic anemia, Cooley anemia, congenital hemolytic anemia, thrombocytopenic purpura, thrombocythemia, macroglobulinemia, cryoglobulinemia, AIDS, HIV, visceral cancers, traumatic injury, solid tumors that respond to immune cell-based therapy, and genetic disorders. In an aspect, the disclosed method can exclude the administration of INF-β.

Thus, disclosed herein are methods of treating a subject in need of hematopoietic cells, comprising administering to a subject an effective amount of hematopoietic cells mobilized in and harvested from a donor, wherein the donor was treated according to a method consisting essentially of administering to the donor an effective amount of FLT3L and a cell adhesion inhibitor, whereby administering the effective amount of FLT3L and the cell adhesion inhibitor mobilizes hematopoietic cells in the donor. In an aspect, a subject is the donor, i.e., the method can be an autologous transplantation. In another aspect, a subject is not the donor, i.e., the method can be an allogeneic transplantation.

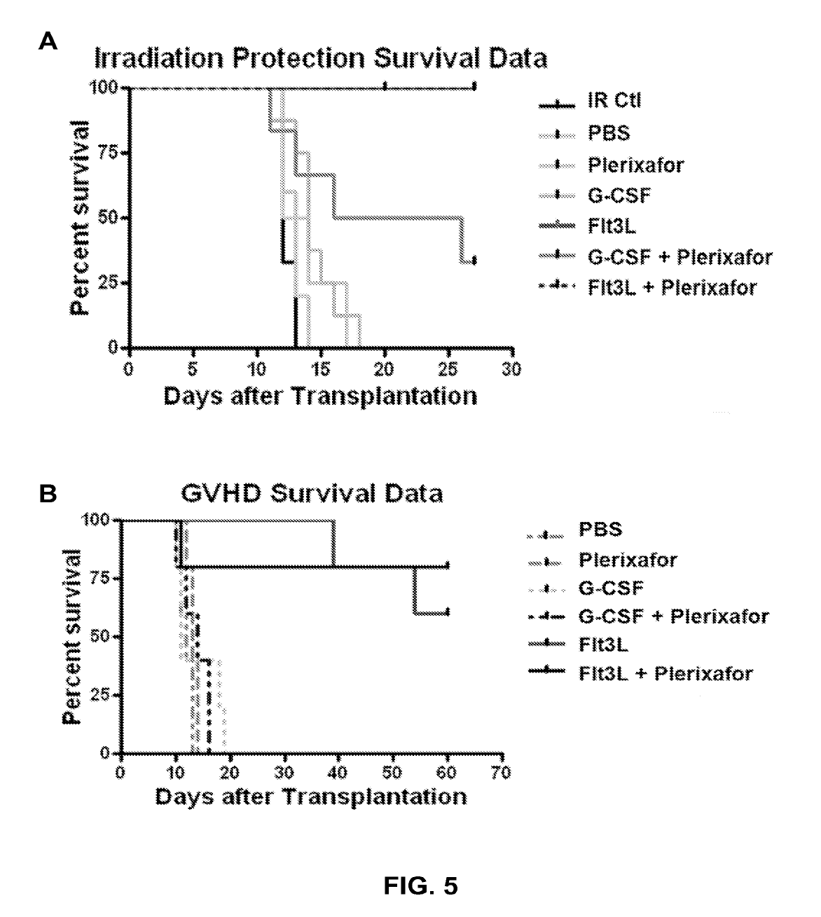

As shown in Example 5, each lethally irradiated mice receiving a transplant of 0.2 million cells obtained according to the disclosed methods were all able to survive for one month (FIG. 5A). By comparison, fewer than 50% of mice transplanted with cells mobilized by G-CSF and Plerixafor, the current clinical standard, survived for one month. Furthermore, in a mouse model of GVHD (each mouse was transplanted with 0.8 million mobilized cells), 80% of mice that received treatment according to the disclosed methods were alive at two months, whereas untreated mice and mice given transplants of cells mobilized by G-CSF and Plerixafor only survived 10-20 days (FIG. 5B).

In an aspect, FLT3L can be administered to a donor once a day for from about 5 days to about 15 days. For example, FLT3L can be administered to a donor once a day for 5, 6, 7, 8, 9, 10, 11, 12, 13, 14 or 15 days, or any time in between. Moreover, FLT3L can be administered to a donor twice a day for from about 5 days to about 15 days. For example, FLT3L can be administered twice a day for 5, 6, 7, 8, 9, 10, 11, 12, 13, 14 or 15 days, or any time in between.

In an aspect, FLT3L can be administered to a donor in an amount from about 0.01 mg/kg to about 1.0 mg/kg, or any amount in between. Thus, the amount of FLT3L administered to a donor in mg/kg can be 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1.0. In an aspect, the amount of FLT3L administered to a donor can be about 0.35 mg/kg.

In an aspect, FLT3L and a cell adhesion inhibitor can be administered contemporaneously. For example, FLT3L can be administered at the same time with or from about 1 hour to about 24 hours before a cell adhesion inhibitor is administered to a donor. Thus, FLT3L can be administered 1, 2, 3, 4, 5, 6, 7, 8, 9, 10, 11, 12, 13, 14, 15, 16, 17, 18, 19, 20, 21, 22, 23 or 24 hours, or any time in between, before a cell adhesion inhibitor is administered to a donor.

In an aspect, FLT3L can be administered from about 1 day to about 15 days before a cell adhesion inhibitor is administered to a donor. For example, FLT3L can be administered 1, 2, 3, 4, 5, 6, 7, 8, 9, 10, 11, 12, 13, 14 or 15 days, or any time in between, before a cell adhesion inhibitor is administered to a donor.

In an aspect, a cell adhesion inhibitor can be one or more of Plerixafor, CTCE-9908, a thiopseudourea compound, TN14003, KRH-2731, KRH-3955, TC14012, AMD070, BKT-140, neutrophil elastase, cathepsin G, and derivatives thereof. It is contemplated that other cell adhesion inhibitors, for example any inhibitor of CXCR4, can be used in the disclosed methods. In an aspect, a cell adhesion inhibitor can be Plerixafor.

In an aspect, Plerixafor can be administered to a donor in an amount from about 0.01 mg/kg to about 1.0 mg/kg, or any amount in between. Thus, the amount of Plerixafor administered to a donor in mg/kg can be 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1.0. In an aspect, the amount of Plerixafor administered to a donor can be about 0.24 mg/kg.

Disclosed herein is a method of mobilizing hematopoietic cells in a subject comprising administering to a subject an effective amount of FLT3L and a cell adhesion inhibitor, whereby administering the effective amount of FLT3L and the cell adhesion inhibitor mobilizes hematopoietic cells in the subject. In an aspect, mobilized hematopoietic cells can be hematopoietic stem cells. Mobilized hematopoietic cells or hematopoietic stem cells are needed in the treatment of many blood disorders as well as other diseases. Blood disorders include, but are not limited to, the disorders described herein. In an aspect, the disclosed method can exclude the administration of INF-β.

In an aspect of a disclosed method of mobilizing hematopoietic cells in a subject, the subject can be a mammal. In an aspect, the mammal can be in need of autologous hematopoietic stem cells or the mammal can be a donor for an allogeneic hematopoietic stem cell transplant. In an aspect, the subject can be a human. In an aspect, the human can be in need of autologous hematopoietic stem cells or the human can be a donor for an allogeneic hematopoietic stem cell transplant.

In an aspect, FLT3L can be administered once a day for from about 5 days to about 15 days or FLT3L can be administered twice a day for from about 5 days to about 15 days. In an aspect, FLT3L call be administered in an amount from about 0.01 mg/kg to about 1.0 mg/kg. Tn an aspect, FLT3L can be administered in an amount of about 0.35 mg/kg.

In an aspect, FLT3L and a cell adhesion inhibitor can be administered contemporaneously. For example, FLT3L can be administered at the same time with or from about 1 hour to about 24 hours before a cell adhesion inhibitor is administered. Thus, in an aspect, FLT3L can be administered 1, 2, 3, 4, 5, 6, 7, 8, 9, 10, 11, 12, 13, 14, 15, 16, 17, 18, 19, 20, 21, 22, 23 or 24 hours, or any time in between, before a cell adhesion inhibitor is administered.

Tn an aspect, FLT3L call be administered from about 1 day to about 15 days before a cell adhesion inhibitor is administered to a donor. For example, FLT3L can be administered 1, 2, 3, 4, 5, 6, 7, 8, 9, 10, 11, 12, 13, 14 or 15 days, or any time in between, before a cell adhesion inhibitor is administered.

In an aspect, the cell adhesion inhibitor can be one or more of Plerixafor, CTCE-9908, a thiopseudourea compound, TN14003, KRH-2731, KRH-3955, TC14012, AMD070, BKT-140, neutrophil elastase, and cathepsin G. In an aspect, the cell adhesion inhibitor can be Plerixafor. In an aspect, Plerixafor can be administered in an amount from about 0.01 mg/kg to about 1.0 mg/kg. In an aspect, the amount of Plerixafor administered in mg/kg can be 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1.0. In an aspect, Plerixafor can be administered in an amount of about 0.24 mg/kg.

In an aspect, the method can comprise measuring the efficacy of FLT3L and/or the cell adhesion inhibitor to mobilize hematopoietic cells in the subject. In an aspect, the method can comprise altering one or more aspects of the administering step and measuring the efficacy of FLT3L and/or the cell adhesion inhibitor to mobilize hematopoietic cells in the subject. For example, in an aspect, depending on whether the administering step mobilizes a sufficient number of hematopoietic cells in the subject, one or more aspects of the administering step can be altered. The skilled person would recognize the aspects of the administering step that could be subject to alterations. Acceptable alterations of the administering step include, but are not limited to, the following: the amount of FLT3L can be altered, the amount of the cell adhesion inhibitor, such as Plerixafor, can be altered, the administering step can be repeated, the site of administration can be altered, the rate of the administering step can be altered (i.e., faster administration or slower administration).

Disclosed herein is a method of mobilizing hematopoietic cells in a cell culture population comprising contacting a cell culture population with an effective amount of FLT3L, whereby contacting the cell culture population with the effective amount of FLT3L mobilizes hematopoietic cells in the cell culture population. In an aspect, the mobilized hematopoietic cells can be hematopoietic stem cells. In an aspect, the cell culture population can comprise one or more of bone marrow cells, embryonic stem cells, umbilical cord blood cells, induced pluripotent stern cells, hematopoietic stern cells, hematopoietic progenitor cells and peripheral blood cells. In an aspect, the disclosed method can exclude the administration of INF-β.

In an aspect, the cell culture population can be contacted with FLT3L once a day for from about 5 days to about 15 days or the cell culture population can be contacted with FLT3L twice a day for from about 5 days to about 15 days. For example, in an aspect, a cell culture population can be contacted with FLT3L twice a day for 5, 6, 7, 8, 9, 10, 11, 12, 13, 14 or 15 days, or any time in between.

In an aspect, the amount of FLT3L can be from about 0.01 μg/mL to about 1.0 μg/mL.

Disclosed herein is a method of treating a subject in need of hematopoietic cells comprising administering to a subject an effective amount of hematopoietic cells mobilized in and harvested from a donor, wherein the donor was treated according to a method comprising administering to the donor an effective amount of FLT3L and a cell adhesion inhibitor, whereby administering the effective amount of FLT3L and the cell adhesion inhibitor mobilizes hematopoietic cells in the donor. In an aspect, the disclosed method can exclude the administration of INF-β.

In an aspect of a disclosed method of treating a subject in need of hematopoietic cells, the subject can be a mammal. In an aspect, the subject can be a human. In an aspect, the donor is a mammal.

In an aspect, FLT3L can be administered to the donor once a day for from about 5 days to about 15 days or FLT3L can be administered to the donor twice a day for from about 5 days to about 15 days. In an aspect, FLT3L can be administered to the donor in an amount from about 0.01 mg/kg to about 1.0 mg/kg. In an aspect, FLT3L can be administered to the donor in an amount of about 0.35 mg/kg.

In an aspect, the cell adhesion inhibitor can be one or more of Plerixafor, CTCE-9908, a thiopseudourea compound, TN14003, KRH-2731, KRH-3955, TC14012, AMD070, BKT-140, neutrophil elastase, and cathepsin G. Tn an aspect, the cell adhesion inhibitor can be Plerixafor. In an aspect, Plerixafor can be administered in an amount from about 0.01 mg/kg to about 1.0 mg/kg. In an aspect, the amount of Plerixafor administered in mg/kg can be 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1.0. Tn an aspect, Plerixafor can be administered in an amount of about 0.24 mg/kg.

Disclosed herein is a method of mobilizing hematopoietic cells in a subject comprising administering to a subject an effective amount of a composition comprising FLT3L, whereby administering the effective amount of the composition mobilizes hematopoietic cells in the subject. In an aspect, the composition can comprise a cell adhesion inhibitor. In an aspect, the mobilized hematopoietic cells can be hematopoietic stem cells. In an aspect, the disclosed method can exclude the administration of INF-β.

In an aspect of a disclosed method of mobilizing hematopoietic cells in a subject, the subject can be a mammal. In an aspect, the mammal can be in need of autologous hematopoietic stem cells or the mammal can be a donor for an allogeneic hematopoietic stem cell transplant. In an aspect of a disclosed method, the subject can be a human. In an aspect, the human can be in need of autologous hematopoietic stem cells or the human can be a donor for an allogeneic hematopoietic stem cell transplant.

In an aspect, the composition can be administered once a day for from about 5 days to about 15 days or FLT3L can be administered twice a day for from about 5 days to about 15 days. In an aspect, the composition can comprise from about 0.01 mg/kg to about 1.0 mg/kg FLT3L. In an aspect, the composition can comprise about 0.35 mg/kg Flt3.

In an aspect, the composition can comprise a cell adhesion inhibitor. In an aspect, the cell adhesion inhibitor can be one or more of Plerixafor, CTCE-9908, a thiopseudourea compound, TN14003, KRH-2731, KRH-3955, TC14012, AMD070, BKT-140, neutrophil elastase, and cathepsin G. In an aspect, the cell adhesion inhibitor can be Plerixafor. In an aspect, the composition can comprise about 0.01 mg/kg to about 1.0 mg/kg Plerixafor. In an aspect, the amount of Plerixafor in mg/kg can be 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1.0. In an aspect, Plerixafor can be in an amount of about 0.24 mg/kg.

In an aspect, the method can comprise measuring the efficacy of the composition to mobilize hematopoietic cells in the subject. In an aspect, the method can comprise altering one or more aspects of one or more administering steps and measuring the efficacy of the composition to mobilize hematopoietic cells in the subject. For example, in an aspect, depending on whether the administering step mobilizes a sufficient number of hematopoietic cells in the subject, one or more aspects of the administering step can be altered. The skilled person would recognize the aspects of the administering step that could be subject to alterations. Acceptable alterations of the administering step include, but are not limited to, the following: the amount of FLT3L can be altered, the amount of the cell adhesion inhibitor, such as Plerixafor, can be altered, the administering step can be repeated, the site of administration can be altered, the rate of the administering step can be altered (i.e., faster administration or slower administration).

Disclosed herein is a method of mobilizing hematopoietic cells in a subject comprising administering to a subject an effective amount of a composition comprising FLT3L and Plerixafor, whereby administering the effective amount of the composition mobilizes hematopoietic cells in the subject. In an aspect, the mobilized hematopoietic cells can be hematopoietic stem cells. In an aspect, the disclosed method can exclude the administration of INF-β.

In an aspect of a disclosed method of mobilizing hematopoietic cells in a subject, the subject can be a mammal. In an aspect, the mammal can be in need of autologous hematopoietic stem cells or the mammal can be a donor for an allogeneic hematopoietic stem cell transplant. In an aspect of a disclosed method, the subject can be a human. In an aspect, the human can be in need of autologous hematopoietic stem cells or the human can be a donor for an allogeneic hematopoietic stem cell transplant.

In an aspect, the composition comprising FLT3L and Plerixafor can be administered once a day or twice a day for from about 5 days to about 15 days. In an aspect, the composition can comprise about 0.01 mg/kg to about 1.0 mg/kg Flt3. In an aspect, the composition can comprise about 0.35 mg/kg Flt3. In an aspect, the composition can comprise about 0.01 mg/kg to about 1.0 mg/kg Plerixafor. In an aspect, the amount of Plerixafor in mg/kg can be 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1.0. In an aspect, Plerixafor can be in an amount of about 0.24 mg/kg.

Disclosed herein is a method of mobilizing hematopoietic cells in a subject comprising (a) administering to a subject an effective amount of a composition comprising FLT3L, and (b) administering to the subject an effective amount of a composition comprising Plerixafor, whereby administering an effective amount of the composition of step (a) and the composition of step (b) mobilize hematopoietic cells in the subject. In an aspect, the mobilized hematopoietic cells can be hematopoietic stem cells. In an aspect, the disclosed method can exclude the administration of INF-β.

In an aspect of a disclosed method of mobilizing hematopoietic cells in a subject, the subject can be a mammal. In an aspect, the mammal can be in need of autologous hematopoietic stem cells or the mammal can be a donor for an allogeneic hematopoietic stem cell transplant. In an aspect of a disclosed method, the subject can be a human. In an aspect, the human can be in need of autologous hematopoietic stem cells or the human can be a donor for an allogeneic hematopoietic stem cell transplant.

In an aspect, the composition comprising FLT3L and the composition comprising Plerixafor can be administered once a day or can be administered twice a day for from about 5 days to about 15 days. In an aspect, the composition can comprise about 0.01 mg/kg to about 1.0 mg/kg Flt3. In an aspect, the composition can comprise about 0.35 mg/kg Flt3. In an aspect, the composition can comprise about 0.01 mg/kg to about 1.0 mg/kg Plerixafor. In an aspect, the amount of Plerixafor in mg/kg can be 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1.0. In an aspect, Plerixafor can be in an amount of about 0.24 mg/kg.

In an aspect, the method can comprise measuring the efficacy of the composition to mobilize hematopoietic cells in the subject. In an aspect, the method can comprise altering one or more aspects of the administering step and measuring the efficacy of the composition to mobilize hematopoietic cells in the subject. For example, in an aspect, depending on whether the administering step mobilizes a sufficient number of hematopoietic cells in the subject, one or more aspects of the administering step can be altered. The skilled person would recognize the aspects of the administering step that could be subject to alterations. Acceptable alterations of the administering step include, but are not limited to, the following: the amount of FLT3L can be altered, the amount of Plerixafor can be altered, one or more administering steps can be repeated, the site of administration can be altered, the rate of the administering step can be altered (i.e., faster administration or slower administration).

Disclosed herein is a method of mobilizing hematopoietic cells in a subject comprising administering to a subject an effective amount of a composition comprising FLT3L, administering to the subject an effective amount of a composition comprising Plerixafor, and mobilizing hematopoietic cells in the subject. In an aspect, the mobilized hematopoietic cells can be hematopoietic stem cells. In an aspect, the disclosed method can exclude the administration of INF-β.

In an aspect of a disclosed method of mobilizing hematopoietic cells in a subject, the subject can be a mammal. In an aspect, the mammal can be in need of autologous hematopoietic stem cells or the mammal can be a donor for an allogeneic hematopoietic stem cell transplant. In an aspect of a disclosed method, the subject can be a human. In an aspect, the human can be in need of autologous hematopoietic stem cells or the human can be a donor for an allogeneic hematopoietic stem cell transplant.

In an aspect of a disclosed method of mobilizing hematopoietic cells in a subject, the composition comprising FLT3L can be administered once a day or can be administered twice a day for from about 5 days to about 15 days. In an aspect, the composition can comprise about 0.01 mg/kg to about 1.0 mg/kg Flt3. In an aspect, the composition can comprise about 0.35 mg/kg Flt3. In an aspect, the composition comprising Plerixafor can be administered once a day or can be administered twice a day for from about 5 days to about 15 days. In an aspect, the composition can comprise about 0.01 mg/kg to about 1.0 mg/kg Plerixafor. In an aspect, the amount of Plerixafor administered in mg/kg can be 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1.0. In an aspect, Plerixafor can be in an amount of about 0.24 mg/kg.

In an aspect, FLT3L and Plerixafor can be administered contemporaneously. For example, FLT3L can be administered at the same time with or from about 1 hour to about 24 hours before Plerixafor is administered. Thus, in an aspect, FLT3L can be administered 1, 2, 3, 4, 5, 6, 7, 8, 9, 10, 11, 12, 13, 14, 15, 16, 17, 18, 19, 20, 21, 22, 23 or 24 hours, or any time in between, before Plerixafor is administered.

In an aspect, FLT3L can be administered from about 1 day to about 15 days before Plerixafor is administered to a donor. For example, FLT3L can be administered 1, 2, 3, 4, 5, 6, 7, 8, 9, 10, 11, 12, 13, 14 or 15 days, or any time in between, before a cell adhesion inhibitor is administered.

In an aspect, the method can comprise measuring the efficacy of one or more compositions to mobilize hematopoietic cells in the subject. In an aspect, the method can comprise altering one or more aspects of one or more administering steps and measuring the efficacy of the compositions to mobilize hematopoietic cells in the subject. For example, in an aspect, depending on whether the administering step mobilizes a sufficient number of hematopoietic cells in the subject, one or more aspects of the administering step can be altered. The skilled person would recognize the aspects of the administering step that could be subject to alterations. Acceptable alterations of the administering step include, but are not limited to, the following: the amount of FLT3L can be altered, the amount of Plerixafor can be altered, the administering step can be repeated, the site of administration can be altered, the rate of the administering step can be altered (i.e., faster administration or slower administration).

Disclosed herein is a method of treating a subject in need of hematopoietic cells comprising administering to a subject an effective amount of hematopoietic cells mobilized in and harvested from a donor, wherein the donor was treated according to a method comprising administering to the donor an effective amount of a composition comprising FLT3L and a cell adhesion inhibitor, whereby administering the composition mobilizes hematopoietic cells in the donor. In an aspect, the mobilized hematopoietic cells can be hematopoietic stem cells. In an aspect, the disclosed method can exclude the administration of INF-β.

In an aspect of a disclosed method of treating a subject in need of hematopoietic cells, the subject can be a mammal. In an aspect, the mammal can be in need of autologous hematopoietic stem cells. In an aspect of a disclosed method, the subject can be a human. In an aspect, the human can be in need of autologous hematopoietic stem cells. In an aspect, the donor can be a mammal. In an aspect, the donor can be a human.

In an aspect, the composition comprising FLT3L can be administered once a day or can be administered twice a day for from about 5 days to about 15 days. In an aspect, the composition call comprise about 0.01 mg/kg to about 1.0 mg/kg F1t3. In all aspect, the composition can comprise about 0.35 mg/kg Flt3.

In an aspect, the composition can comprise a cell adhesion inhibitor. In an aspect, a cell adhesion inhibitor can be one or more of Plerixafor, CTCE-9908, a thiopseudourea compound, TN14003, KRH-2731, KRH-3955, TC14012, AMD070, BKT-140, neutrophil elastase, and cathepsin G. In an aspect, the cell adhesion inhibitor can be Plerixafor. In an aspect, the composition can comprise about 0.01 mg/kg to about 1.0 mg/kg Plerixafor. In an aspect, the composition can comprise about 0.24 mg/kg Plerixafor. In an aspect, the amount of Plerixafor in mg/kg can be 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1.0.

In an aspect, the method can comprise measuring the efficacy of the composition to mobilize hematopoietic cells in the subject. In an aspect, the method can comprise altering one or more aspects of the administering step and measuring the efficacy of the composition to mobilize hematopoietic cells in the subject. For example, in an aspect, depending on whether the administering step mobilizes a sufficient number of hematopoietic cells in the subject, one or more aspects of the administering step can be altered. The skilled person would recognize the aspects of the administering step that could be subject to alterations. Acceptable alterations of the administering step include, but are not limited to, the following: the amount of FLT3L can be altered, the amount of the cell adhesion inhibitor, such as Plerixafor, can be altered, the administering step can be repeated, the site of administration can be altered, the rate of the administering step can be altered (i.e., faster administration or slower administration).

Disclosed herein is a composition comprising FLT3L and a cell adhesion inhibitor. Disclosed herein is a composition comprising FLT3L and Plerixafor. Disclosed herein is a composition comprising a FLT3L and one or more cell adhesion inhibitors. Disclosed herein is a composition comprising FLT3L, Plerixafor, and at least one other cell adhesion inhibitor. In an aspect, the disclosed composition can exclude INF-β. In an aspect, a disclosed composition can comprise a pharmaceutically acceptable carrier.

All patents, patent applications, and other scientific or technical writings referred to anywhere herein are incorporated by reference in their entirety. The invention illustratively described herein suitably can be practiced in the absence of any element or elements, limitation or limitations that are not specifically disclosed herein. Thus, for example, in each instance herein any of the terms “comprising”, “consisting essentially of”, and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by embodiments, optional features, modification and variation of the concepts herein disclosed can be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

### EXAMPLES

The following examples are put forth so as to provide those of ordinary skill in the art with a complete disclosure and description of how the compounds, compositions, and methods claimed herein are used and evaluated and are intended to be purely exemplary of the invention and are not intended to limit the scope of what the inventors regard as their invention. However, those of skill in the art should, in light of the present disclosure, appreciate that many changes can be made in the specific aspects which are disclosed and still obtain a like or similar result without departing from the spirit and scope of the invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### Combination of FLT3L and Plerixafor Synergistically Mobilized More HSCs and Committed Progenitors Than the Combination of G-CSF and Plerixafor in Mice

Since HSCs are important for long-term reconstitution and progenitor cells are critical for short-term rapid reconstitution in transplantation, whether a combination of FLT3L and Plerixafor can mobilize both types of cells was investigated. In mice, Lin⁻c-kit⁺Sca-1⁺ cells are enriched HSCs and Lin⁻c-kit⁺Sca-1⁻ cells are more committed progenitor cells. Flow cytometric analysis showed that FLT3L administration for 5 days increased only Lin-c-kit+Sca-1-progenitor cells but not HSCs, while administration of 7 days and 10 days increased both Lin-c-kit+Sca-1-progenitor cells (0.47% vs. 1.8%, n=3, p<0.01) and Lin⁻c-kit⁺Sca-1⁻ HSC (0.13% vs. 1.8%, n=3, p<0.01) (FIG. 1). FLT3L and Plerixafor acted synergistically to increase HSCs and progenitor cells significantly when compared with each alone. Furthermore, the mobilization effect of the FLT3L and Plerixafor combination was much stronger than the G-CSF and Plerixafor combination, the strongest mobilizer currently used clinically (5.9% vs. 2%, 10.3% vs. 1%, n=3, p<0.01, respectively). The flow results were validated by another method, stem cell colony formation assay. These data indicate that FLT3L in combination with Plerixafor was an effective stem/progenitor cell mobilizer.

In FIG. 1, WT B6D2F1/J mice were treated with FLT3L or G-CSF for 10 or 5 days, respectively, followed by a one-hour Plerixafor treatment. PBS served as a control. On the last day of the treatment, mice were sacrificed and peripheral blood cells were subjected to flow cytometry to determine percentages (among total WBC) of stem-cell enriched (Lin⁻CD117⁺Sca-1⁺) (FIG. 1A) or more committed precursor cells (Lin⁻c-kit⁻Sca-1⁺) (FIG. 1B).

### Example 2

### Combination of FLT3L and Plerixafor Enhanced NK Cell Expansion

NK cells were dramatically increased in both the peripheral blood and the spleen when mice were treated with FLT3L alone for 14 days. With or without combination of Plerixafor, a similar in vivo NK cell expansion was noted when FLT3L was administered only for 7 or 10 days (FIG. 2; FLT3L or FLT3L plus Plerixafor vs. PBS; gated on total white blood cells). Further, FLT3L alone and the combination of FLT3L with Plerixafor generated much higher NK cell content when compared with the combination of GCSF and Plerixafor (FIG. 2). Of note, rapid lymphocyte and NK recovery after transplantation has been shown to result in a lower rate of relapse, and NK cells have been reported to suppress GVHD in a mouse model.

In FIG. 2, WT C57BL/6 or B6D2F1/J mice were treated with FLT3L or G-CSF for 10 or 5 days, respectively, followed by a one-hour Plerixafor treatment. PBS served as a control. Peripheral blood cells were counted using trypan blue and assayed by flow cytometry to examine percentages of NK cells (NK1.1⁺CD3⁻). FIG. 2A shows data for one representative experiment and FIG. 2B shows summary data for three experiments.

### Example 3

### Combination of FLT3L and Plerixafor Decreased CD8⁺ T Cells

Plerixafor alone or in combination with G-CSF moderately gave rise to a proportional increase of CD8+ T cells, whereas FLT3L alone or in combination with Plerixafor dramatically decreased this subset of cells (FIG. 3). It is known that donor CD8⁺ T cells are mainly responsible for GVHD in HSCT, and G-CSF is considered a toxic mobilizer as it also induces GVHD. These data show that the combination of FLT3L and Plerixafor is a better than expected mobilization regimen to reduce GVHD.

In FIG. 3, WT B6D2F1/J mice were treated with FLT3L or G-CSF for 10 or 5 days, respectively, followed by a one-hour Plerixafor treatment. PBS served as a control. Peripheral blood cells were assayed by flow cytometry to examine percentages of CD8⁺ T cells (CD3⁻ CD8⁺) in total white blood cells. Data were summarized from a group of three mice.

### Example 4

### Co-Administration of Cytokines and FLT3L

The co-administration of cytokines is common in other types of cell mobilization methods. However, the data show that the administration of cytokines was contraindicated for the purposes of mobilizing HSCs and progenitor cells. FIG. 4 shows that when IFN-β, a routinely utilized cytokine, was co-administered with GM-CSF or FLT3L, the percentage of LSK cells was dramatically reduced, and considerably fewer cells were mobilized. LSK cells are hematopoietic stem cells that were Lin-negative, Scal -positive, and c-kit-negative.

### Example 5

### Peripheral Blood Mobilized by the Disclosed Methods Prolongs Survival In Mouse Models

Peripheral blood (PB) mobilized by FLT3L and Plerixafor prolongs mouse survival in a lethally irradiated syngeneic mouse model and a fully mismatched acute GVHD model. To test whether the mobilized stem cells and other immune cells can improve transplantation, mobilized cells were administered to lethally irradiated (IR) syngeneic mice at the dose of 0.2 million PB cells for each recipient mouse. The cells mobilized by either FLT3L alone or its combination with Plerixafor can fully protect mice from death, whereas other regimens do not have any protective effects except that of the combination of G-CSF and Plerixafor, which has a partial protective effect (FIG. 5A). In a fully mismatched GVHD model, the mice treated with cells mobilized by the combination of FLT3L and Plerixafor had an 80% survival rate two months after transplantation, whereas all mice died in other groups between 10-20 days after transplantation except in the FLT3L alone group, in which 60% of mice survived up to day 60 (FIG. 5B).

### Example 6

### Combination of FLT3L and Plerixafor Increased Hematopoietic Stem Cell Mobilization and Improved Transplantation Outcome

Hematopoietic stem cell (HSC) transplantation has curative potential for patients with hematological malignancies. Clinically, HSCs derived from mobilized peripheral blood are used more frequently than bone marrow. However, the currently used mobilizing agents yield grafts that often do contain an appropriate number of HSCs. As described herein, FLT3L (F) synergized with Plerixafor (P) to mobilize phenotypically defined HSCs. Mobilization with the combination of FLT3L and Plerixafor (FP or FP combination) yielded superior results when compared to mobilization with granulocyte colony-stimulating factor (G-CSF) alone and when compared to mobilization with the combination of G-CSF with Plerixafor (GP or GP combination).The data presented herein show that the FP combination mobilized more regulatory T cells, natural killer cells, and plasmacytoid dendritic cells when compared with mobilization with G-CSF alone or when compared to mobilization with the GP combination. Both syngeneic and allogeneic grafts mobilized by the FP combination led to long-term survival in transplanted mice.

Hematological malignancies contribute to almost 10% of the mortalities caused by cancers in the United States. (Siegel et al., 2013) Hematopoietic stem cell transplantation (HSCT) is a well-established treatment with curative potential for a variety of hematological cancers. In addition to bone marrow transplantation (BMT), mobilized peripheral blood provides a rich source of HSCs for transplantation. Successful HSCT depends on a mobilization regimen (i) that provides an adequate number of HSCs for engraftment and (ii) is less likely to provoke transplant-related complications. Current mobilization regimens often do not mobilize sufficient numbers of cells into the peripheral blood, or the mobilized cells often lead to post-transplant complications, such as graft-versus-host disease (GVHD). In mice, GVHD is characterized by a hunched back, inactivity, diarrhea, ruffled fur, and a weight loss greater than 10%.

The CXCR4 antagonist Plerixafor (Mozobil) induces safe and rapid mobilization of clinical grafts capable of promoting robust hematopoietic recovery in HSCT patients (Devine et al., 2008). However, the number of hematopoietic stem/progenitor cells (CD34⁺ cells) mobilized by Plerixafor alone is less than the number of cells mobilized by granulocyte colony-stimulating factor (GCSF). GCSF is considered the current gold standard for a mobilizing regimen.

Flt3 ligand (FLT3L) is a stem-cell specific growth factor that expands and mobilizes stem cells in mice following 10 day-administration either as (i) a single agent or (ii) in combination with other molecules such as IL-8 and G-CSF (Brasel et al., 1997; de Kruijf et al., 2010). When administered for 5 or 10 consecutive days, FLT3L mobilized CD34⁺ HSCs in healthy donors (Anandasabapathy et al., 2013).

As described herein, the effect of the combination of two clinical grade drugs (i.e., FLT3L and Plerixafor) on stem cell mobilization was evaluated. The fate of these mobilized cells in both syngeneic and allogeneic transplantation murine models was also examined. These data demonstrate that the FP combination (as compared to other treatment regimens) mobilized stem cells and other cell subsets to obtain a better engraftment and a prolonged survival following both syngeneic and allogeneic transplantation.

In the experiments described herein, the following materials and methods were utilized.

8 to 10 weeks old BALB/c and C57BL/6 mice were purchased from the National Cancer Institute (Frederick, Md.).

C57BL/6 mice were injected intraperitoneally (i.p.) either (i) with recombinant human FLT3L (Celldex Therapeutics or Miltenyi Biotec) (350 mg/kg/day) for 10 consecutive days or (ii) with G-CSF (Amgen) (150 mg/kg/day) for 5 consecutive days. The capacity of human FLT3L to bind murine FLT3L receptors and Plerixafor to block murine Cxcr4 receptor was previously reported. (See, e.g., Lyman et al., 1994; Brawel et al., 1996; Gerlach et al., 2001; Broxmeyer et al., 2005). Twelve hours after the last dose of either FLT3L or G-CSF, the mice were injected i.p. with either PBS or Plerixafor (AMD3100, Sigma) (5 mg/kg). The mice were euthanized one hour later.

As described by Ferraro et al., 2011, peripheral blood was collected retro-orbitally while the mice were under deep anesthesia. Briefly, the mice were euthanized by an i.p. injection with 100 μL of a mixture of ketamine (54.3 mg/mL) and xylazine (9.1 mg/mL). Blood was collected from the retro-orbital venous plexus through a microcapillary heparinized tube. The mice were then euthanized while still deeply anesthetized. RBCs were depleted by RBC lysis buffer. The remaining cells were subjected to flow cytometric analysis or transplantation.

For syngeneic transplantation, C57BL/6 mice were subjected to 9 Gy of irradiation delivered in 2 fractions separated by 4 hours using RS 2000 X-Ray Q2 Irradiator (Rad Source Technologies, Suwanee GP). Four hours later, each mouse was transplanted via the tail vein with 2×10⁵ mobilized RBC-depleted peripheral blood cells from C57BL/6 mice. For allogeneic transplantation, each recipient BALB/c mouse was subjected to a lethal irradiation (delivered in 2 fractions separated by 4 hours using RS 2000 X-Ray Q2 Irradiator) and was then injected with 8×10⁵ mobilized RBC depleted peripheral blood cells isolated from C57BL/6 mice. The cell numbers used for these transplantation studies was based on a showing that about 90% of mice survived when syngeneically infused with 1×10⁶ peripheral nucleated cells mobilized by G-CSF (Glass et al., 1997; Zeng et al., 1997), but only around 17% of mice survived if 2.5×10⁵ cells mobilized by G-CSF were transplanted (Zeng et al., 1997). Therefore, in the syngeneic transplant experiments described herein, 2×10⁵ mobilized cells were used to whether treatment with a combination of FLT3L and Plerixafor (i.e., FP or the FP combination) mobilized more cells than treatment with GCSF alone. Considering the likely graft rejection and GVHD complication in the allogeneic model, a 4-fold higher number of cells (i.e., 8×10⁵ donor cells isolated from C57BL/6 mice) were administered intravenously (i.v.) into each BALB/c recipient for allogeneic transplants. The lethally irradiated mice were housed in a sterile environment and baytril antibiotic was provided in the drinking water to prevent infection-caused mortality. After transplantation, mice were monitored daily and the phenotypes of the mice were recorded.

Colony formation assays were performed using MethoCult GF M3434 (StemCell Technologies, Vancouver, British Columbia, Canada) according to the manufacturer's instructions. Briefly, 4.6×10⁴ of mobilized WBCs were re-suspended in 0.3 mL Iscove's modified Dulbecco's media (Life Technologies, Grand Island, N.Y.) with 2% FBS and added to 3 mL MethoCult. The vortexed mixture was then dispensed into three 35-mm dishes at 1.1 mL per plate, which were placed into a 100-mm culture dish and incubated at 37° C. at 5% CO₂ for 12 days. Colonies were counted on an inverted microscope (Carl Zeiss Microscopy Thornwood, N.Y.).

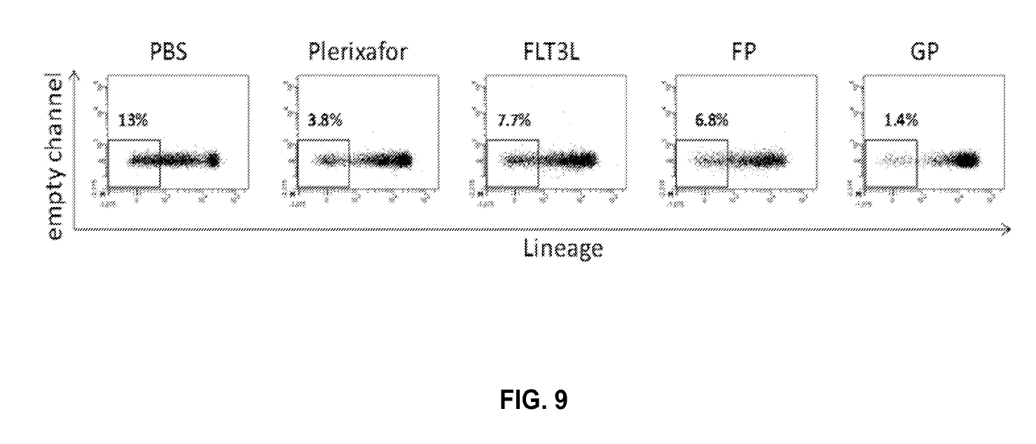

The mAbs reactive with murine cell antigens were purchased from BD Biosciences or eBioscience. Cell preparation and analysis were performed as described by Yu et al., 2010. Mouse HSCs were defined as Lin⁻Sca-1⁻c-Kit⁺ (LSK) cells, and Lin⁻ (lineage negative) cells were gated as shown in FIG. 9. The immune subsets were gated as CD3⁻NK1.1⁺ for natural killer (NK) cells, CD3⁺CD25⁺Foxp3⁺ for regulatory T cells (Tregs), B220⁻CD11c⁺CD11b⁻ for conventional dendritic cells (cDCs), and CD11b⁻CD11c^{low}PDCA1⁺SiglecH⁺ for plasmacytoid DCs (pDCs).

For continuous endpoints, Student's t-test was used to compare two independent groups. Measurements such as frequency and absolute number LSK cells, colony formation, and absolute number of Tregs were logarithmically transformed to normalize the data and stabilize the variance. A 1-way ANOVA model was applied to multiple comparisons, and a 2-way ANOVA model was performed to evaluate the synergistic effect between FLT3L and Plerixafor. The log-rank test was used to compare survival curves. All tests were 2-sided. P values were adjusted for multiple comparisons by Holms' procedure. p<0.05 was considered as statistically significant.

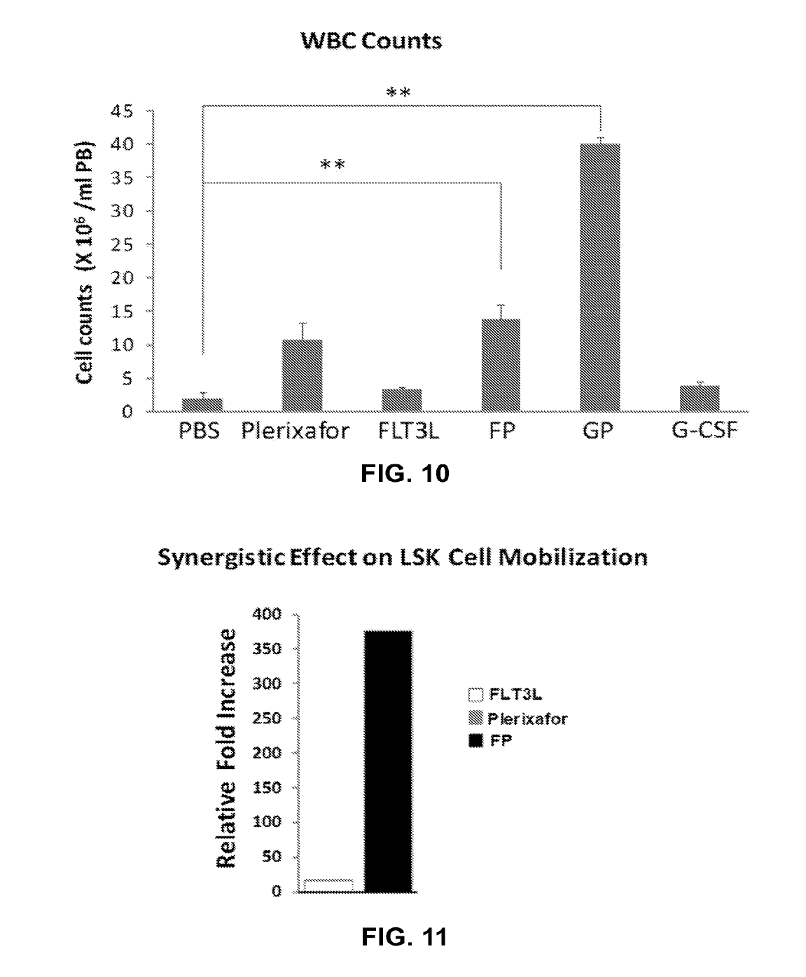

The data provided herein demonstrate that the FP combination effectively mobilized LSK cells into peripheral blood. To test the effect of the combination of FLT3L and Plerixafor (FP or the FP combination) on HSC mobilization, both the WBC counts and HSCs (Lin⁻Sca-1⁺c-Kit⁺, LSK cells, a hematopoietic stem cell-enriched population) in the peripheral blood were analyzed in mice. The mice were subjected to various mobilization regimens, including the combination of G-CSF and Plerixafor (GP or the GP combination). Cell counts showed that the mice receiving the GP combination had the highest WBC counts and that the mice receiving the FP combination the second highest WBC counts (FIG. 10).

In FIG. 10, mice were treated with different mobilizing regimens and total white blood cells were enumerated by microscopic examination of blood after red blood cell lysis. The combination of G-CSF and Plerixafor (GP) regimen led to highest number of white blood cells in peripheral blood after mobilization. Shown are the summarized data from 3 mice in one of two experiments with similar data. Error bars represent S.D. ** indicates p<0.01.

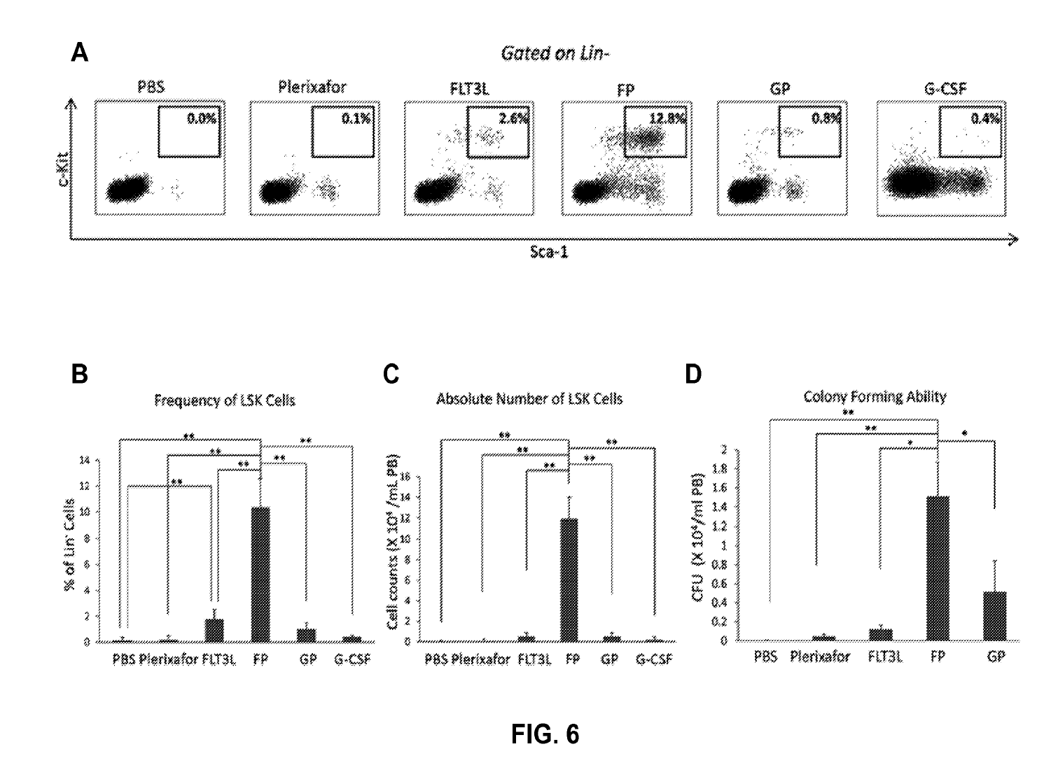

Compared with PBS-treated mice, Plerixafor-treated mice generated a moderate increase in the number LSK cells. When compared to PBS-treated mice, FLT3L-treated mice mobilized significantly more LSK cells into the blood (FIG. 6A). However, the FP combination mobilized cells with a higher frequency and mobilized a greater total number of LSK cells. For example, when compared to FLT3L alone, the FP combination had a 6-fold greater frequency of cell mobilization and a 12-fold greater number of mobilized LSK cells. (FIGS. 6B and C). The FP combination showed a synergistic mobilizing effect. (p<0.05, FIG. 11).

In FIG. 11, the absolute amount of LSK cells mobilized into peripheral blood was determined. The increase of LSK cells by each mobilization regimen is presented as the fold ratio to PBS control. The paired bars compare the additive effect of FLT3L alone and of Plerixafor alone (left, composite bar) versus the effect of the FP combination (right, black bar). A two-way ANOVA model was applied to evaluate the synergistic effect of FLT3L and Plerixafor. p<0.05 was obtained for the synergistic effect test.

In FIG. 6, peripheral blood cells in mice treated with indicated mobilizing regimens were harvested, subjected to RBC depletion, and stained for HSCs (Lin⁻Sca-1⁺c-Kit⁺, LSK cells). The representative data from 1 of 9 mice with similar results are shown in FIG. 6A, and the summary data of 3 mice in 1 of 3 experiments with similar data for the frequency (FIG. 6B) and the absolute number (FIG. 6B) of LSK cells are also shown. FIG. 6D shows mobilized peripheral blood cells subjected to colony formation assay. Shown are the summary data of 3 mice for the colony forming units larger than 200 cells. For all panels, *p<0.05, **p<0.01, and error bars represent standard deviation.

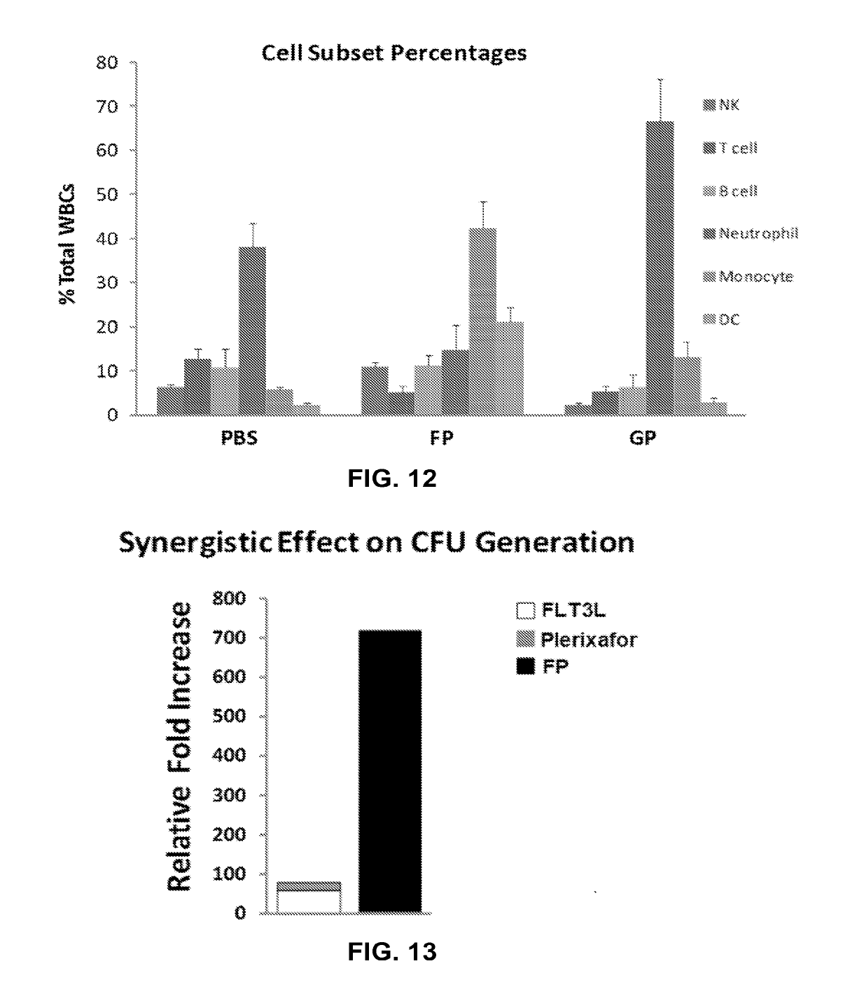

Although the mice treated by the GP combination had the highest WBC counts, the absolute number of LSK cells was similar to that in mice treated with FLT3L alone. (FIG. 12). When compared to treatment with G-CSF alone, the FP combination mobilized a significantly higher number of LSK cells. (FIG. 6A-C, p<0.05). Colony formation assays revealed that cells mobilized by the FP combination contained significantly more colony-forming units than any other treatment regimen, including the GP combination, FLT3L alone, and Plerixafor alone. (FIG. 6D). A synergistic effect was also observed between FLT3L and Plerixafor (FIG. 13, p<0.01).

In FIG. 12, the constitution of immune cell subsets in mobilized peripheral blood was determined by flow cytometric analysis with corresponding antibodies (B cell: CD3⁻CD19⁺, T cell: CD3⁺NK1.1⁻; NK cell: CD3⁻NK1.1⁺; Neutrophil: Gr1^{high}CD11b⁺; Monocyte: Gr1^{med}CD11b⁺; DC: CD11c⁺). Shown are the summarized data from 3 mice in one of two experiments with similar data. Error bars represent S.D.

In FIG. 13, the numbers of colony forming units in mobilized blood were determined. The increase of CFU by each mobilization regimen is presented as the fold ratio to PBS control. The paired bars compare the additive effect of FLT3L alone and of Plerixafor alone (left, composite bar) versus the effect of the FP combination (right, black bar). A two-way ANOVA model was applied to evaluate the synergistic effect of the combination of FLT3L and Plerixafor. p<0.001 was obtained for the synergistic effect test.

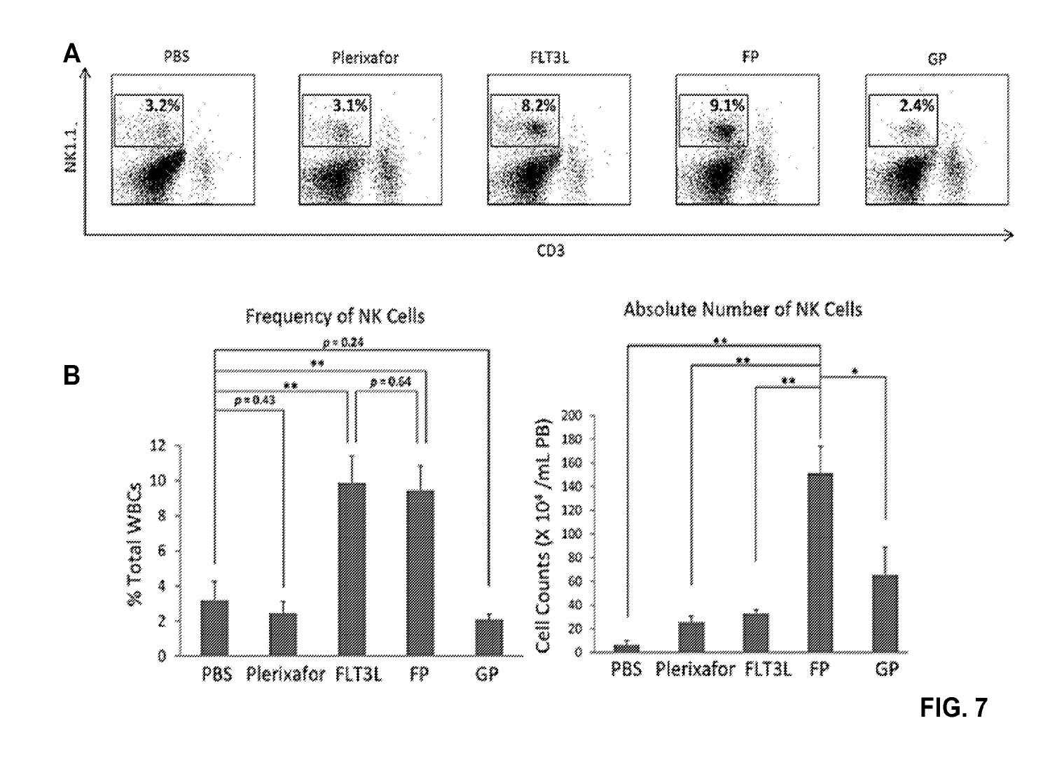

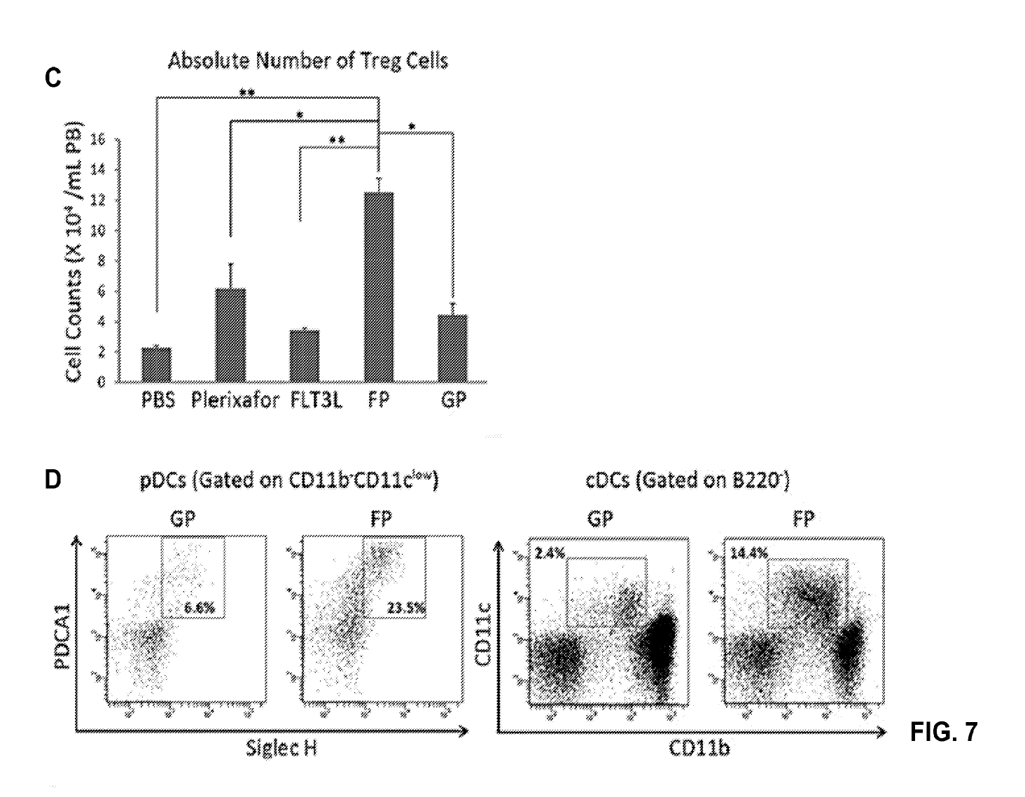

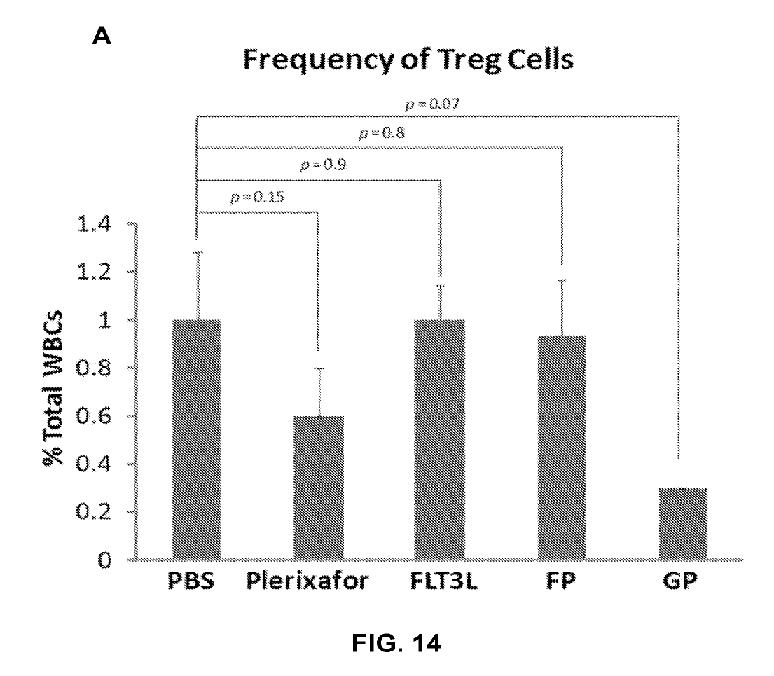

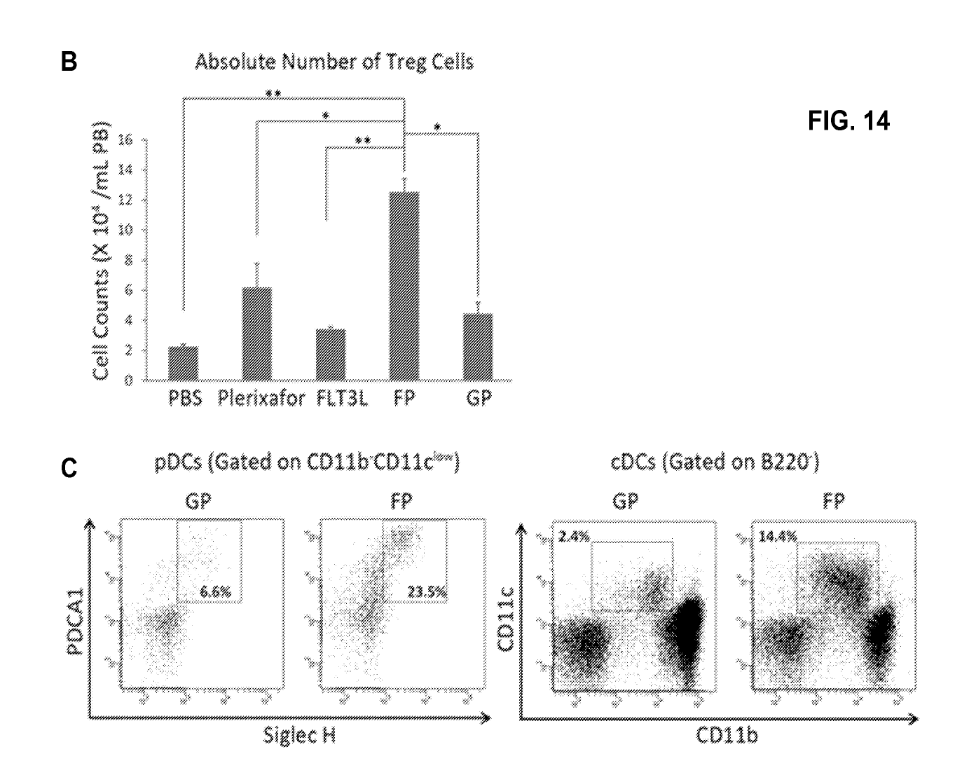

The data presented herein demonstrate that the FP combination effectively mobilized NK cells, Tregs, and DCs into peripheral blood. In addition to LSK cells, the effect of these treatments on other immune cell subsets was assessed. Mobilization with the FP combination or FLT3L alone significantly increased NK (CD3⁻NK1.1⁺) cell percentages to the same extent. (FIGS. 7A and 7B) The administration of the FP combination increased the absolute number of NK cells. This increase exceeded that of any other treatment regimen FLT3L alone. (FIG. 7B, right side panel) Although none of the mobilizing agents induced a significant increase of Treg frequency (FIG. 14), the FP combination led to a significantly higher absolute number of Tregs in mobilized blood when compared to the increase achieved by any other treatment regimen. (FIG. 7C) The data show that FLT3L expands DCs both in vivo and in vitro (Maraskovsky et al., 1996; Strobl et al., 1997). The 2 major subsets of DCs (i.e., pDCs and cDCs) were both significantly increased in proportion following treatment with the FP combination as compared to GP. (FIG. 7D)

In FIG. 7, peripheral blood cells in mice treated with indicated mobilizing regimens were harvested, subjected to RBC depletion, stained for NK cells, and subjected to flow cytometric analysis. FIG. 7A shows representative data and FIG. 7B shows summary data of 3 mice in 1 of 3 experiments with similar results. FIG. 7C shows mobilized peripheral blood cells subjected to flow cytometric analysis for Tregs. Shown are the summary data of 3 mice for the absolute number of Tregs. FIG. 7D shows GP-mobilized peripheral blood cells from 3 mice (a pooled sampled) and FP-mobilized peripheral blood cells from 3 mice (a pooled sampled) and stained for cDCs (B220⁻CD11b⁺CD11c⁺) and pDCs (CD11b⁻CD11c^{low}PDCA⁺SiglecH⁺). For all panels, *p<0.05, **p<0.01, and error bars represent standard deviation.

In FIG. 14A, mice were treated with different mobilizing regimens and total white blood cells were stained and subjected to flow cytometric analysis of Treg cells. None of the regimens increased Treg cell percentages in mobilized peripheral blood (right panel). Shown are the summarized data from 3 mice. Error bars represent S.D. FIG. 14B shows that the combination of FLT3L and Plerixafor effectively mobilized Treg cells (absolute cell number) and FIG. 14C shows mobilization of DCs into peripheral blood. Peripheral blood cells in mice treated with indicated mobilizing regimens were harvested, subjected to red blood cell depletion, and subjected to flow cytometric analysis for Treg cells. Shown are the summary data of 3 mice for the absolute number of Treg cells. GP-mobilized peripheral blood cells from 3 mice were pooled together and FP-mobilized peripheral blood cells from 3 mice were pooled together and each was stained for cDCs (B220⁻CD11b⁺CD11c⁺) and pDCs (CD11b⁻CD11c^{low}PDCA⁺SiglecH For all panels, * indicates p<0.05, ** indicates p<0.01, and error bars represent S.D.

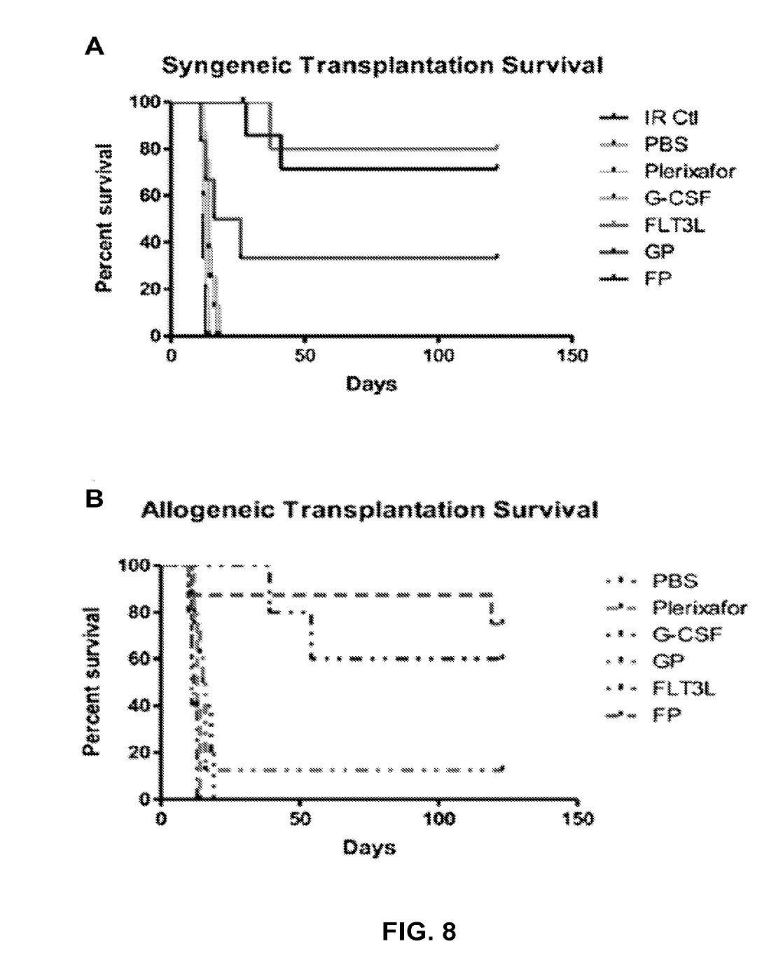

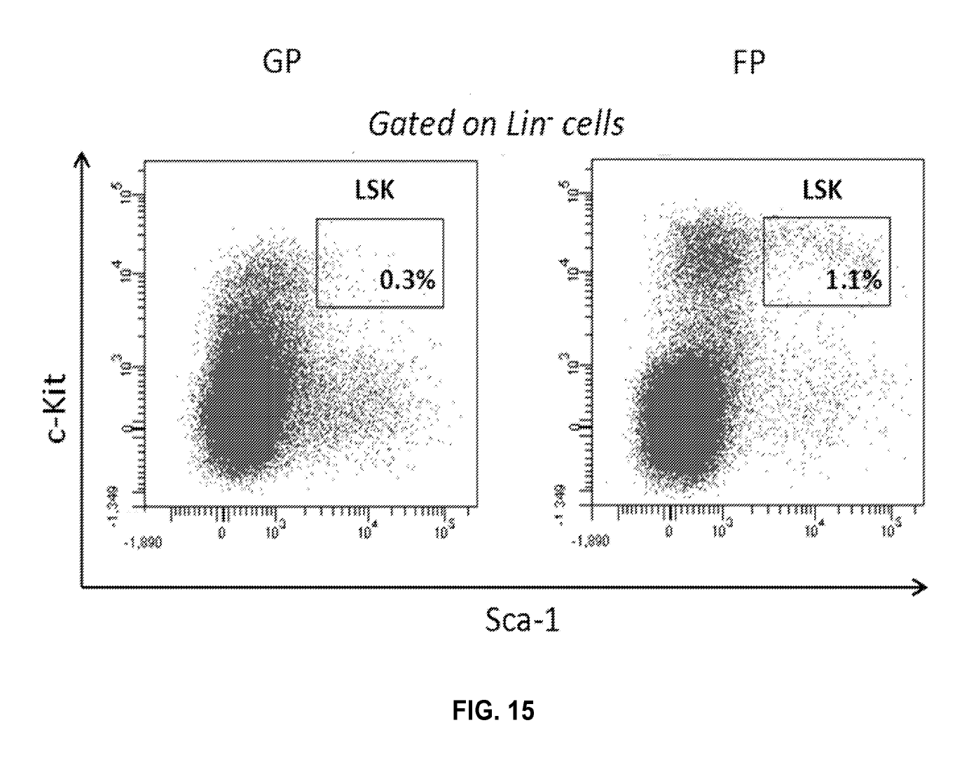

The data discussed herein show that the FP-mobilized grafts significantly enhanced survival of mice in both syngeneic and allogeneic settings. To test the clinical potential of grafts mobilized by the FP combination, cells mobilized by different regimens were transfused into lethally irradiated syngeneic mice. Mice receiving grafts mobilized by PBS, Plerixafor alone, or G-CSF alone died within 21 days. In these three groups (i.e., PBS alone, Plerixafor alone, or G-CSF alone), the frequency of progenitor cells or LSK cells were much lower than in the group receiving the FP combination. (FIG. 6A) However, mice receiving grafts either mobilized with FLT3L or the FP combination had a 100% survival rate at day 21. Four months after transplantation, approximately 70% of the mice receiving grafts either mobilized with FLT3L or the FP combination were still alive. (FIG. 8A) The engraftment of the cells mobilized by the FP combination was significantly higher than the number of cells mobilized by the combination (p<0.05). The mice having received a GP mobilized graft had a survival rate of only 35% at 4 months post-transplantation. An examination of bone marrow in the surviving mice also showed that mice receiving the FP-mobilized grafts contained a higher number of LSK cells than those mice receiving GP-mobilized grafts. (FIG. 15)

In FIG. 15, lethally irradiated C57BL/6 mice were transplanted with grafts mobilized by different regimens. At four months post-transplantation, the surviving mice that received grafts mobilized by the combination of FLT3L and Plerixafor (FP) or the combination of G-CSF and Plerixafor (GP) were sacrificed. The HSCs in the bone marrow were determined using flow cytometry. Shown are representative data from 1 out of 3 mice with similar results. Syngeneic mice receiving grafts mobilized by FP generated more HSCs in bone marrow than the mice receiving grafts from mobilized by GP.

In FIG. 8, survival analysis of lethally irradiated C57BL/6 mice (A) or BALB/c mice (B) receiving peripheral blood cells from C57BL/6 mice mobilized by the indicated regimens. Irradiated mice without any cell infusion (IR Ctl) served as a control group. A total of 2×10⁵ (A) or 8×10⁵ (B) peripheral blood cells were transplanted for each recipient. Each group contains 5 to 8 mice. A log-rank test was used to compare survival curves. P values in (A) are as follows: FP versus GP (p<0.05), FP versus FLT3L (p=0.53), FP versus G-CSF (p<0.01), FP versus Plerixafor (p<0.001), and FP versus PBS (p<0.001). P values in (B) are as follows: FP versus GP (p<0.05), FP versus FLT3L (p=0.41), FP versus G-CSF (p<0.01), FP versus Plerixafor (p<0.01), and FP versus PBS (p<0.001).

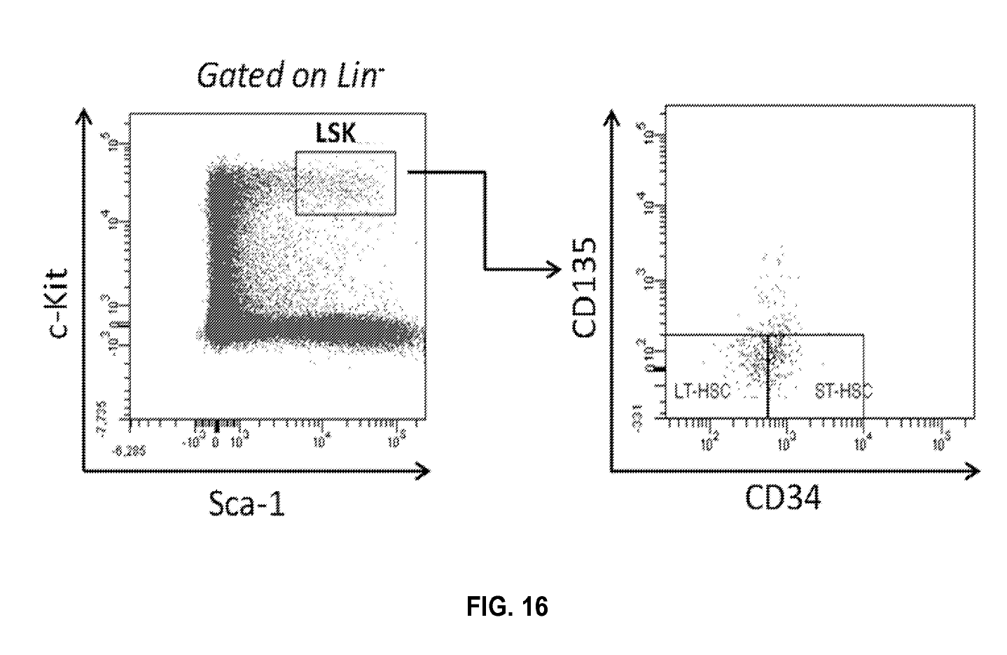

Next, the transplantation efficacy of the FP-mobilized cells in an allogeneic transplantation model was examined. Lethally irradiated BALB/c mice were transplanted with peripheral blood cells mobilized by the different regimens from C57BL/6 mice. More mobilized peripheral blood cells were transplanted in this MHC mismatched model than in the syngeneic model described above (i.e., 8×10⁵ (syngeneic) versus 2×10⁵ cells (mismatched) per recipient). All mice receiving a PBS-mobilized graft, a Plerixafor-mobilized graft, or a G-CSF-mobilized graft died within 3 weeks after transplantation. Conversely, 80% of mice receiving FP-mobilized grafts and 66% of mice receiving FLT3L-mobilized grafts survived to 4 months post-transplantation. (FIG. 8B) The survival rate of the FP-mobilized group was significantly higher than that observed in the GP-mobilized group (i.e., 12.5% alive at 4 months post-transplantation, p<0.05). At 4 months post-transplantation, the mice in the FP-mobilized group that remained healthy were euthanized. In the bone marrow of these mice, there were both long-term HSCs (CD135⁻CD34⁻ LSK) and short-term HSCs (CD135⁻CD34⁺ LSK) (Yang et al., 2005) which originated from donor C57BL/6 mice (H2K^{b}). (FIG. 16).

In FIG. 16, lethally irradiated BALB/c mice were transplanted with grafts from C57BL/6 mice that were mobilized by the indicated regimens. At four months post-transplantation, the surviving mice were sacrificed. The long-term HSCs (LT-HSCs, defined as Lin⁻Sca-1⁺c-Kit⁺CD34⁻ CD135⁻) as well as the short-term HSCs (ST-HSCs, defined as Lin⁻Sca-1⁺c-Kit⁺CD34⁺CD135⁻) in the bone marrow were determined by flow cytometric analysis. All cells were gated on H2K^{b} (C57BL/6 source). Shown are representative data from 1 out of 5 mice with similar results.

GCSF-based regimens (alone or in combination with chemotherapy or other agents) are the most common methods used to mobilize HSCs for autologous transplantation. However, poor mobilization of these cells is a pressing problem. In a study of 976 patients who went through mobilization, 29.7% failed to achieve sufficient numbers of stem cells (Gertz et al., 2010). As described herein, treatment with the combination of FP induced a significantly more robust HSC mobilization when compared with the most potent clinic regimen currently available (i.e., the combination of GP). When compared to the GP combination, the FP combination led to a 6-fold increase in frequency and 12-fold increase in absolute number of phenotypically defined stern cells mobilized into peripheral blood. (FIG. 6) The data provided herein confirm a synergistic effect between FLT3L (F) and Plerixafor (P). The lethally irradiated syngeneic mice were rescued with a small amount of FP-mobilized grafts. (FIG. 8A)

An allogeneic transplantation requires a sufficient number of HSCs as well as other cell subsets to help overcome allogeneic transplantation-related complications, such as GVHD and engraftment failure due to allograft rejection. FP-mobilized grafts significantly improved the survival of mice following an allogeneic transplantation. (FIG. 8B) The FP combination mobilized more HSCs, NK cells, Tregs, and DCs into peripheral blood, which suppressed acute GVHD. NK cells induce an antitumor response while suppressing the development of GVHD in mice (Olson et al., 2010). Tregs are important immune regulatory cells that generate protective effects against GVHD after allogeneic transplantation (Rezvani et al., 2006). Although cDCs are generally considered to enhance GVHD (Markey et al., 2009), the simultaneous increase of pDCs may attenuate the effect of cDCs because pDCs have been shown to inhibit GVHD (Toubai et al., 2010; Banovic et al., 2009). In the allogeneic model described herein, mice transplanted with FP-mobilized peripheral blood cells by had a lower incidence of acute GVHD than mice transplanted with PBS-mobilized peripheral blood cells, G-CSF mobilized peripheral blood cells, and GP-mobilized peripheral blood cells.

When compared to the other treatment regimens, the FP combination consistently generated higher numbers of monocytes (Gr1^{med}CD11b⁺) (FIG. 12), which is a cell population reported to be involved in both autologous (Kerryn Ansell 2013) and allogeneic (Mougiakakos et al., 2013) stem cell transplantation as monocytic myeloid-derived suppressor cells (Gabrilovich et al., 2009). In allogeneic stem cell transplantation, myeloid-derived suppressor cells, like NK cells and Tregs, also mitigate GHVD.

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

### REFERENCES

Anandasabapathy N, et al. (2013) A phase 1 trial of the hematopoietic growth factor CDX-301 (rhuFLT3L) in healthy volunteers. Biol Blood Marrow Transplant. 19:S112-S113.

Banovic T, et al. (2009) Graft-versus-host disease prevents the maturation of plasmacytoid dendritic cells. J Immunol. 182:912-920.

Brasel K, et al. (1997) Flt3 ligand synergizes with granulocyte-macrophage colony-stimulating factor or granulocyte colony-stimulating factor to mobilize hematopoietic progenitor cells into the peripheral blood of mice. Blood. 90:3781-3788.

Brasel K, et al. (1996) Hematologic effects of flt3 ligand in vivo in mice. Blood. 88:2004-2012.

Broxmeyer H E, et al. (2005) Rapid mobilization of murine and human hematopoietic stem and progenitor cells with AMD3100, a CXCR4 antagonist. J Exp Med. 201:1307-1318.

de Kruijf E J, et al. (2010) Hematopoietic stem and progenitor cells are differentially mobilized depending on the duration of Flt3-ligand administration. Haematologica. 95:1061-1067.

Devine S M, et al. (2008) Rapid mobilization of functional donor hematopoietic cells without G-CSF using AMD3100, an antagonist of the CXCR4/SDF-1 interaction. Blood. 112:990-998.

Ferraro F, et al. (2011) Diabetes impairs hematopoietic stem cell mobilization by altering niche function. Sci Transl Med. 3:104ra101.

Gabrilovich D I, et al. (2009) Myeloid-derived suppressor cells as regulators of the immune system. Nat Rev Immunol. 9:162-174.1.

Gerlach L O, et al. (2001) Molecular interactions of cyclam and bicyclam non-peptide antagonists with the CXCR4 chemokine receptor. J Biol Chem. 276:14153-14160.

Gertz M A. (2010) Current status of stem cell mobilization. Br J Haematol. 150:647-662.

Glass B, et al. (1997) Allogeneic peripheral blood progenitor cell transplantation in a murine model: evidence for an improved graft-versus-leukemia effect. Blood. 90:1694-1700.

Guimond M, et al. (2010) In vivo role of Flt3 ligand and dendritic cells in NK cell homeostasis. J Immunol. 184:2769-2775.

Kerryn Ansell LFP. (2013) Autograft monocytes: the bad humors of autologous peripheral blood hematopoietic stem cell transplantation. J Stem Cell Res Ther. S3:5.

Kessans M R, et al. (2010) Plerixafor: a peripheral blood stem cell mobilizer. Pharmacotherapy 30:485-92.

Korbling M, et al. (2011) Twenty-five years of peripheral blood stem cell transplantation. Blood 117:6411-6.

Lyman S D, et al. (1994) Cloning of the human homologue of the murine flt3 ligand: a growth factor for early hematopoietic progenitor cells. Blood. 83:2795-2801

Maraskovsky E, et al. (1996) Dramatic increase in the numbers of functionally mature dendritic cells in Flt3 ligand-treated mice: multiple dendritic cell subpopulations identified. J Exp Med. 184:1953-1962.

Markey K A, et al. (2009) Conventional dendritic cells are the critical donor APC presenting alloantigen after experimental bone marrow transplantation. Blood. 113:5644-5649.

Mohty M, et al. (2011) The role of plerixafor in optimizing peripheral blood stem cell mobilization for autologous stem cell transplantation. Leukemia 25:1-6.

Mougiakakos D, et al. (2013) Immunosuppressive CD14 [symbol] HLA-DRlow/neg IDO [symbol] myeloid cells in patients following allogeneic hematopoietic stem cell transplantation. Leukemia. 27:377-388.

Okada S, et al. (1992) In vivo and in vitro stem cell function of c-kit- and Sca-1-positive murine hematopoietic cells. Blood 80:3044-3050.

Olson J A, et al. (2010) NK cells mediate reduction of GVHD by inhibiting activated, alloreactive T cells while retaining GVT effects. Blood. 115:4293-4301. Rezvani K, et al. (1994) High donor FOXP3-positive regulatory T-cell (Treg) content is associated with a low risk of GVHD following HLA-matched allogeneic SCT. Blood. 108:1291-1297.

Savani B N, et al. (2007) Absolute lymphocyte count on day 30 is a surrogate for robust hematopoietic recovery and strongly predicts outcome after T cell-depleted allogeneic stem cell transplantation. Biol Blood Marrow Transplant 13:1216-23.

Schmitz N, et al. (1996) Randomised trial of filgrast immobilised peripheral blood progenitor cell transplantation versus autologous bone-marrow transplantation in lymphoma patients. Lancet. 347:353-357.

Schrezenmeier H, et al. (2007) Worse outcome and more chronic GVHD with peripheral blood progenitor cells than bone marrow in HLA-matched sibling donor transplants for young patients with severe acquired aplastic anemia. Blood 110:1397-400.

Siegel R, et al. (2013) Cancer statistics, 2013. CA Cancer J Clin. 63:11-30.

Strobl H, et al. (1997) flt3 ligand in cooperation with transforming growth factor-betal potentiates in vitro development of Langerhans-type dendritic cells and allows single-cell dendritic cell cluster formation under serum-free conditions. Blood. 90:1425-1434.

Tanhehco Y C et al. (2010) Plerixafor mobilization leads to a lower ratio of CD34⁺ cells to total nucleated cells which results in greater storage costs. J Clin Apher 25:202-8.

Toubai T, et al. (2010) Immunization with host-type CD8 {alpha} [symbol] dendritic cells reduces experimental acute GVHD in an TL-10-dependent manner. Blood. 115:724-735.

Waller E K, et al. (2011) Larger Numbers of Donor Naïve CD8+ T-Cells and Plasmacytoid Dendritic Cell Precursors In Allogeneic BM Grafts From Unrelated Donors Are Associated with Improved Survival: Results From BMT CTN 0201. Blood 118(21): 459-460.

Yang L, et al. (2005) Identification of Lin(−)Sca1( [symbol] )kit( [symbol] ) CD34( [symbol] )Flt3-short-term hematopoietic stem cells capable of rapidly reconstituting and rescuing myeloablated transplant recipients. Blood. 105:2717-2723.

Yu J, et al. (2010) CD94 surface density identifies a functional intermediary between the CD56bright and CD56dim human NK-cell subsets. Blood. 115:274-281.

Zeng D, et al. (1997) Granulocyte colony-stimulating factor reduces the capacity of blood mononuclear cells to induce graft-versus-host disease: impact on blood progenitor cell transplantation. Blood. 90:453-463.

What is claimed is:

1. A method of treating a subject in need of hematopoietic cells comprising:
administering to a subject an effective amount of hematopoietic cells mobilized in and harvested from a donor, wherein the donor was treated according to a method comprising administering to the donor an effective amount of FLT3L and a cell adhesion inhibitor, whereby administering the effective amount of FLT3L and the cell adhesion inhibitor mobilizes hematopoietic cells in the donor.

2. The method of claim 1, wherein the subject is a mammal.

3. The method of claim 2, wherein the mammal is a human.

4. The method of claim 1, wherein the donor is a mammal.
